(19) United States
(12) Patent Application Publication
(10) Pub. No.: US 2006/0159616 A1
(43) Pub. Date: Jul. 20, 2006

(54) Methods for detecting endocrine cancer

(75) Inventors: George Yousef, St.John's (CA); Elefttherious P Diamandis, Toronto (CA)

Correspondence Address: MERCHANT & GOULD PC, P.O. BOX 2903, MINNEAPOLIS, MN, 55402-0903, US

(73) Assignee: Mount Sinai Hospital, Toronto, ON (CA)

(21) Appl. No.: 10/526,111

(22) PCT Filed: Aug. 28, 2003
(86) PCT No.: PCT/CA03/01311

§ 371 Date: Nov. 22, 2005

Related U.S. Application Data
(60) Provisional application No. 60/407,332, filed on Aug. 28, 2002.

(51) Int. Cl. A61K 49/00; A61K 51/00; G01N 33/574

(52) U.S. Cl. 424/1.11; 435/7.23; 424/9.6

(57) ABSTRACT

Kallikrein 13 constitutes a novel biomarker for diagnosis, treatment and monitoring of endocrine cancer. A method is provided for diagnosing and monitoring endocrine cancer or a predisposition to endocrine cancer, in a subject comprising detecting hK13 in a sample from the subject. Imaging methods, screening methods for test agents for inhibiting breast or ovarian cancer, and therapeutic applications based on kallikrein 13 are also described.

### FIELD OF THE INVENTION

The invention relates to methods for detecting endocrine cancer.

### BACKGROUND OF THE INVENTION

Epithelial ovarian cancer is the most lethal gynaecologic malignancy, constituting approximately 90% of all ovarian cancer cases (Jemal, A. et al, 2003; Auerspreg, N. 1998). The high mortality rate is usually ascribed to late diagnosis, since epithelial ovarian tumors commonly lack early warning symptoms. Furthermore, ovarian carcinomas often lack definite precursor lesions, are quite heterogeneous and the molecular pathways underlying their progression are still elusive. Thus, many attempts have been made to predict the biology of ovarian tumors, in order to determine prognosis and develop individualized treatment strategies. The International Federation of Gynecology and Obstetrics (FIGO) stage at diagnosis represents the major prognostic factor in ovarian cancer. FIGO stage I patients have a 5-year survival of 80-90% compared with only 15-20% for women with stage III and IV disease (Schink J C, 1999). Other well established conventional prognostic markers include tumor grade, patient age, residual tumor after surgery, presence and absence of ascites and histology (Trope, C. 1998; Eisenhauer, E A et al, 1999).

In addition to these clinicopathological parameters, numerous tumor markers with prognostic potential have also been identified, including DNA ploidy, oncogenes, cell cycle regulatory proteins and inhibitors, enzymes, growth factors, extracellular matrix components and proteases (Ozalp S et al., 2001; Milde-Langosch K et al, 2003; Masciullo, V et al, 2000; Gotlieb W H et al, 2001; Shen G H et al, 2000; Hata K et al, 2001; Schmalfeldt B et al, 2001). More recently, cDNA microarray analyses and bioinformatic approaches such as serial analysis of gene expression (SAGE), have also been used to identify genes differentially expressed in ovarian cancer, in order to subcategorize tumours on the basis of molecular profile, often unveiling important biological, diagnostic and prognostic information (Schummer M. et al, 1999; Welsh J B et al, 2001; Haviv, I and Campbell I G, 2002; Sawiris G P et al, 2002; and Hough C D et al, 2000).

Among the newly identified prognostic factors are human tissue kallikreins, a group of serine proteases encoded by 15 structurally similar, hormonally-regulated genes, clustered in tandem on chromosome 19q13.4 (Yousef G M and Diamandis E P, 2001). Accumulating evidence indicates that at least 11 kallikrein family members are differentially expressed in ovarian cancer at both the mRNA and protein levels and several demonstrate clinical utility as prognostic biomarkers (Yousef G M and Diamandis E P, 2002; Diamandis E P and Yousef G M, 2002). Moreover, the serum levels of kallikrein proteins, hK6, hK10 and hK11, are elevated in a proportion of ovarian cancer patients and as such, they may represent putative serological screening and/or diagnostic biomarkers for ovarian cancer (Diamandis E P et al, 2000; Diamandis, E P et al, 2003; Luo L Y et al, 2001; Luo et al, 2003; Diamandis, E P et al, 2002).

Human kallikrein gene 13 (KLK13, previously known as KLK-L4), recently discovered by the positional candidate cloning approach, is a novel androgen/progestin-regulated serine protease gene, predominantly expressed in endocrine tissues, including the prostate, testis and breast (Yousef G M et al, 2000). Preliminary evidence indicates that KLK13 is implicated in hormone-dependent malignancies and has clinical utility. For one, KLK13 is down-regulated, at the mRNA level, in breast cancer tissues and cell lines (Yousef G M et al, 2000). A subsequent and extensive quantitative RT-PCR study demonstrated that KLK13 expression in breast tumor tissues is an indicator of favourable prognosis, since patients with KLK13-positive tumors exhibit a longer PFS and OS (Chang, A et al, 2002). Furthermore, five testis-specific splice variants of the KLK13 gene were identified and found to be down-regulated in testicular tissues compared to the matched normal counterparts (Chang, A et al, 2001).

The citation of any reference herein is not an admission that such reference is available as prior art to the instant invention.

### SUMMARY OF THE INVENTION

Applicants have developed highly sensitive and specific procedures for quantification of kallikrein 13 in biological fluids and tissues. The kallikrein 13 protein was expressed in yeast, purified to homogeneity, monoclonal and polyclonal antibodies were generated, and an immunofluorometric method was developed for kallikrein 13 quantification.

The kallikrein 13 immunoassay has a detection limit of 0.05 μg/L. The assay is specific for kallikrein 13 and has no cross-reactivity with other homologous kallikreins. The assay is linear from 0 to 20 μg/L; within-run and between-run CVs were <10%. Kallikrein 13 was found to be expressed in various tissues including esophagus, tonsil, trachea, lung, cervix and prostate. Kallikrein 13 was also found in seminal plasma, amniotic fluid, follicular fluid, ascites of ovarian cancer patients, breast milk and cytosolic extracts of ovarian cancer tissues. Kallikrein 13 was immunohistochemically localized in both normal and cancerous prostate. Kallikrein 13 was found to be overexpressed in ovarian tumor tissues compared to normal. Using an ELISA, kallikrein 13 levels were quantified in ovarian tumor extracts and correlated with clinicopathological variables and outcome {progression-free survival (PFS), overall survival (OS)}. Patients with kallikrein 13 positive tumors most often had early stage (Stage I/II) disease, no residual tumor after surgery and optimal debulking success. Patients with kallikrein 13 positive tumors had a significantly longer PFS and OS than kallikrein 13 negative patients.

Therefore, kallikrein 13 constitutes a new biomarker for diagnosis and monitoring (i.e. monitoring progression or therapeutic treatment) of endocrine cancer, in particular ovarian cancer. In accordance with an aspect of the invention kallikrein 13 is used for the diagnosis, monitoring, and prognosis of endocrine cancer, and it may be used as a biomarker before surgery or after relapse. In another aspect of the invention, kallikrein 13 is used for the diagnosis, monitoring, and prognosis of endocrine tumors.

Kallikrein 13, and agents that bind to kallikrein 13, may be used to detect endocrine cancer and they can be used in the diagnostic evaluation of endocrine cancer, and the identification of subjects with a predisposition to such disorders. Methods for detecting kallikrein 13 can be used to monitor endocrine cancer.

The presence of kallikrein 13 in a sample can be assessed, for example, by detecting the presence in the sample of (a) kallikrein 13 protein or fragments thereof; or (b) metabolites which are produced directly or indirectly by a kallikrein 13 protein.

In an embodiment, the invention provides a method for detecting a kallikrein 13 protein comprising (a) obtaining a sample from a patient; (O) detecting or identifying in the sample a kallikrein 13 protein; and (c) comparing the detected amount with an amount detected for a standard.

In an aspect of the invention, a method for screening a subject for endocrine cancer is provided comprising (a) obtaining a biological sample from a subject; (b) detecting the amount of kallikrein 13 in said sample; and (c) comparing said amount of kallikrein 13 detected to a predetermined standard, where detection of a level of kallikrein 13 which is significantly different than that of a standard indicates disease.

The term “detect” or “detecting” includes assaying, imaging or otherwise establishing the presence or absence of the target kallikrein 13, subunits thereof, or combinations of reagent bound targets, and the like, or assaying for, imaging, ascertaining, establishing, or otherwise determining one or more factual characteristics of endocrine cancer, metastasis, stage, outcome, or similar conditions. The term encompasses diagnostic, prognostic, and monitoring applications for kallikrein 13.

According to a method involving a kallikrein 13 protein the levels in a sample from a patient are compared with the normal levels of kallkrein 13 protein in samples of the same type obtained from controls (e.g. samples from individuals not afflicted with disease). Significantly altered levels in the sample of the kallikrein 13 protein relative to the normal levels in a control is indicative of disease. Thus, a method is provided of assessing whether a patient is afflicted with or has a pre-disposition to endocrine cancer, comprising comparing (a) levels of kallikrein 13 in a sample from the patient; and (b) normal levels of kallikrein 13 in samples of the same type obtained from control patients not afflicted with endocrine cancer, wherein significantly altered levels of kallikrein 13 relative to the corresponding normal levels of kallikrein 13, is an indication that the patient is afflicted with or has a pre-disposition to endocrine cancer.

A significant difference between the levels of kallikrein 13 in the patient and the normal levels is an indication that the patient is afflicted with or has a predisposition to endocrine cancer.

The invention further relates to a method of assessing the efficacy of a therapy for endocrine cancer in a patient. A method of the invention comprises comparing: (a) levels of kallikrein 13 in a sample from the patient obtained from the patient prior to providing at least a portion of the therapy to the patient; and (b) levels of kallikrein 13 in a second sample obtained from the patient following therapy.

A significant difference between the levels of kallikrein 13 in the second sample relative to the first sample is an indication that the therapy is efficacious for inhibiting endocrine cancer.

The “therapy” may be any therapy for treating endocrine cancer including but not limited to therapeutics, radiation, immunotherapy, gene therapy, and surgical removal of tissue. Therefore, the method can be used to evaluate a patient before, during, and after therapy.

In an aspect, the invention provides a method for monitoring the progression of endocrine cancer in a patient the method comprising:
(a) detecting kallikrein 13 in a sample from the patient at a first time point;
(b) repeating step (a) at a subsequent point in time; and
(c) comparing the levels detected in (a) and (b), and therefrom monitoring the progression of the endocrine cancer.

The invention also provides a method for assessing the potential efficacy of a test agent for inhibiting endocrine cancer, and a method of selecting an agent for inhibiting endocrine cancer.

The invention also contemplates a method of assessing the potential of a test compound to contribute to endocrine cancer comprising:
(a) maintaining separate aliquots of endocrine cancer diseased cells in the presence and absence of the test compound; and
(b) comparing the level of kallikrein 13 in each of the aliquots.

A significant difference between the levels of kallikrein 13 in the aliquot maintained in the presence of (or exposed to) the test compound relative to the aliquot maintained in the absence of the test compound, indicates that the test compound potentially contributes to endocrine cancer.

The present invention also relates to a method for diagnosing and monitoring endocrine carcinoma in a subject comprising measuring kallikrein 13 in a sample from the subject Kallikrein 13 may be measured using a reagent that detects kallikrein 13 preferably antibodies specifically reactive with kallikrein 13 or a part thereof.

In an aspect of the invention, a method for screening a subject for endocrine cancer is provided comprising (a) obtaining a biological sample from a subject; (b) detecting the amount of kallikrein 13 in said sample; and (c) comparing said amount of kallikrein 13 detected to a predetermined standard, where detection of a level of kallikrein 13 greater than that of a standard indicates disease.

In an embodiment, the invention relates to a method for diagnosing and monitoring endocrine cancer in a subject by quantitating kallikrein 13 in a biological sample from the subject comprising (a) reacting the biological sample with an antibody specific for kallikrein 13 which is directly or indirectly labelled with a detectable substance; and (b) detecting the detectable substance.

In another aspect the invention provides a method for using an antibody to detect expression of a kallikrein 13 protein in a sample, the method comprising: (a) combining an antibody specific for kallikrein 13 with a sample under conditions which allow the formation of antibody:protein complexes; and (b) detecting complex formation, wherein complex formation indicates expression of the protein in the sample. Expression may be compared with standards and is diagnostic of endocrine cancer.

Embodiments of the methods of the invention involve (a) reacting a biological sample from a subject with an antibody specific for kallikrein 13 which is directly or indirectly labelled with an enzyme; (b) adding a substrate for the enzyme wherein the substrate is selected so that the substrate, or a reaction product of the enzyme and substrate forms fluorescent complexes; (c) quantitating kallikrein 13 in the sample by measuring fluorescence of the fluorescent complexes; and (d) comparing the quantitated levels to levels obtained for other samples from the subject patient, or control subjects. In an embodiment the quantitated levels are compared to levels quantitated for control subjects without endocrine cancer wherein an increase in kallikrein 13 levels compared with the control subjects is indicative of endocrine cancer.

A particular embodiment of the invention comprises the following steps
(a) incubating a biological sample with a first antibody specific for kallikrein 13 which is directly or indirectly labeled with a detectable substance, and a second antibody specific for kallikrein 13 which is immobilized;
(b) detecting the detectable substance thereby quantitating kallikrein 13 in the biological sample; and
(c) comparing the quantitated kallikrein 13 with levels for a predetermined standard.

The standard may correspond to levels quantitated for samples from control subjects without endocrine cancer, with a different disease stage (e.g. early stage), or from other samples of the subject. Increased levels of kallikrein 13 as compared to a standard may be indicative of cancer.

In a particular aspect of the invention, increased levels as compared to a standard (e.g. kallikrein 13 negative tumors) are indicative of one or more of the following: early stage ovarian cancer (Stage I/II), no residual tumors, optimal debulking success, tumors characterized by clear cell and mucinous histotypes, and longer PFS and OS (i.e. lower risk of relapse and death).

The invention also contemplates the methods described herein using multiple markers for endocrine cancer. Therefore, the invention contemplates a method for anaylzing a biological sample for the presence of kallikrein 13 and other markers that are specific indicators of endocrine cancer. Other markers include kallikrein 2, kallikrein 3, kallikrein 4, kallikrein 5, kallikrein 6, kallikrein 7, kallikrein 8, kallikrein 9, kallikrein 10, kallikrein 11, kallikrein 14, and kallikrein 15; human stratum corneum chymotryptic enzyme (HSCCE), haptoglobin alpha, osteopontin, CA125, CA15-3, CA19-9, OVX1, lysophosphatidic acid (LPA), carcinoembryonic antigen (CEA), prostate specific antigen, prostate specific membrane antigen, and prostate stem cell antigen. Preferably the other markers are markers to kallikreins. In an aspect of the invention, the markers are one or more of hK8, hK9, hK11, and hK14, or hK4, hK5, hK6, bK7, hK10, and hK15, or hK6, hK10, and hK11. The methods described herein may be modified by including reagents to detect the markers, or nucleic acids for the markers.

The invention provides an isolated and purified kallikrein 13 polypeptide that is non-glycosylated and has a molecular weight of about 25 to 30 kDa, in particular 28 kDa. The invention also provides an isolated and purified kallikrein 13 polypeptide that is glycosylated and has a molecular weight of about 50 kDa.

In accordance with an aspect of the invention a method is provided comprising administering to a subject an agent that has been constructed to target one or more kallikreins.

The invention also contemplates a method comprising administering to cells or tissues imaging agents that carry labels for imaging and bind to kallikrein 13 and optionally other markers of endocrine cancer, and then imaging the cells or tissues.

The invention further contemplates an in vivo method comprising administering to a mammal one or more imaging agent that carries a label for imaging and binds to kallikrein 13 and optionally other markers of endocrine cancer, and then imaging the mammal.

According to a particular aspect of the invention, an in vivo method for imaging endocrine cancer is provided comprising:
(a) injecting a patient with an imaging agent that binds to kallikrein 13, the agent carrying a label for imaging the endocrine cancer,
(b) allowing the imaging agent to incubate in vivo and bind to kallikrein 13 associated with the endocrine cancer; and
(c) detecting the presence of the label localized to the endocrine cancer.

In an embodiment of the invention the imaging agent is an antibody which recognizes kallikrein 13. In another embodiment of the invention the imaging agent is a chemical entity which recognizes kallikrein 13.

The imaging agent carries a label to image kallikrein 13 and other markers of endocrine cancer. Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for endocrine cancer. For example, an imaging method may further comprise injecting the patient with one or more of an agent that binds to human stratum corneum chymotryptic enzyme (HSCCE), kallikrein 2, kallikrein 3, kallikrein 4, kallikrein 5, kallikrein 6, kallikrein 7, kallikrein 8, kallikrein 9, kallikrein 10, kallikrein 11, kallikrein 14, kallikrein 15, CA125, CA15-3, CA19-9, OVX1, lysophosphatidic acid (LPA), carcinoembryonic antigen (CEA), prostate specific antigen, prostate specific membrane antigen, or prostate stem cell antigen.

The invention also relates to kits for carrying out the methods of the invention. In an embodiment, the kit is for assessing whether a patient is afflicted with endocrine cancer, and it comprises reagents for assessing kallikrein 13. In another embodiment, the kit is for determining the prognosis of a patient afflicted with endocrine cancer, and it comprises reagents for assessing kallikrein 13.

In another aspect, the invention relates to a kit for assessing the suitability of each of a plurality of test compounds for inhibiting endocrine cancer in a patient. The kit comprises reagents for assessing kallikrein 13 and optionally a plurality of test agents or compounds.

The invention contemplates a kit for assessing the presence of endocrine cancer cells, wherein the kit comprises antibodies specific for kallikrein 13, and optionally antibodies specific for other markers associated with endocrine cancer.

Additionally the invention provides a kit for assessing the potential of a test compound to contribute to endocrine cancer. The kit comprises endocrine cancer cells and reagents for assessing kallikrein 13, and optionally other markers associated with endocrine cancer.

The invention provides a diagnostic composition comprising kallikrein 13 or agents that bind to kallikrein 13 or parts thereof. Agents can be labeled with detectable substances.

The invention also relates to therapeutic applications for endocrine cancer.

In an aspect, the invention relates to compositions comprising a kallikrein 13 or part thereof, or an antibody specific for kallikrein 13, and a pharmaceutically acceptable carrier, excipient, or diluent. A method for treating or preventing endocrine cancer in a patient is also provided comprising administering to a patient in need thereof, a kallikrein 13 protein or part thereof, an antibody specific for kallikrein 13, or a composition of the invention. In an aspect the invention provides a method of treating a patient afflicted with or at risk of developing endocrine cancer comprising inhibiting expression of kallikrein 13.

In an aspect, the invention provides antibodies specific for kallikrein 13 that can be used therapeutically to destroy or inhibit the growth of kallikrein 13 expressing cancer cells, (e.g. ovarian cancer or breast cancer cells), or to block kallikrein 13 activity. In addition, kallikrein 13 proteins may be used in various immunotherapeutic methods to promote immune-mediated destruction or growth inhibition of tumors expressing kallikrein 13.

The invention also contemplates a method of using kallikrein 13 or part thereof, or an antibody specific for kallikrein 13 in the preparation or manufacture of a medicament for the prevention or treatment of endocrine cancer.

Another aspect of the invention is the use of kallikrein 13, peptides derived therefrom, or chemically produced (synthetic) peptides, or any combination of these molecules, for use in the preparation of vaccines to prevent endocrine cancer and/or to treat endocrine cancer.

The invention contemplates vaccines for stimulating or enhancing in a subject to whom the vaccine is administered production of antibodies directed against kallikrein 13.

The invention also provides a method for stimulating or enhancing in a subject production of antibodies directed against kallikrein 13. The method comprises administering to the subject a vaccine of the invention in a dose effective for stimulating or enhancing production of the antibodies.

The invention further provides a method for treating, preventing, or delaying recurrence of endocrine cancer. The methods comprise administering to the subject a vaccine of the invention in a dose effective for treating, preventing, or delaying recurrence of endocrine cancer.

The invention also contemplates the methods, compositions, and kits described herein using additional markers associated with endocrine cancer. The methods described herein may be modified by including reagents to detect the additional markers, or nucleic acids for the markers.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 illustrates the production of recombinant hK13 protein with the Picihia pastoris yeast expression system. The proteins were separated on SDS-PAGE and detected with an hK13 peptide antibody developed in-house. Lane 1, molecular mass marker; lanes 2-6, X-33 yeast strain producing KLK13 after 1-6 days of induction with 5% methanol; lane 7, X-33 strain as above but without methanol induction; lane 8, X-33 strain without transfected KLK13 cDNA and no methanol induction; lane 9, X-33 strain without transfected KLK13 cDNA and 3 days of methanol induction. Note the presence of highly glycosylated (˜50 kDa) hK13 and non-glycosylated hK13 (28 kDa) in lanes 2-6.

FIG. 2 shows the results of SDS-PAGE electrophoresis of purified recombinant hK13 before and after treatment with PNGaseF diglycosylation enzyme. Panel A: Lane 1: Positive control (horseradish peroxidase). Lane 2: Negative control (soyabean trypsin inhibitor). Lane 3: recombinant hK13 (identified by arrow). Lane 4: Recombinant hK13 after treatment with PNGaseF. The higher molecular weight band represents PNGaseF and the lower molecular weight band (˜28 kDa) represents deglycosylated hK13. Panel B: The gel wag stained with acidic fuchsin sulfite, a glycoprotein stain (Pierce). Note the staining of the glycoproteins horseradish peroxidase (positive control) and hK13 in Lanes 1 and 3. M, molecular weight markers.

FIG. 3 shows a typical calibration curve for the hK13 immunoassay. The background fluorescence (zero calibrator) has been subtracted from all measurements. The dynamic range of the assay is 0.05-20 μg/L.

FIG. 4 illustrates the tissue expression of hK13 protein. Human tissues were pulverized and cytosolic extracts prepared, as described in the Example. The concentration of hK13 was then measured with the ELISA assay. Results were normalized for total protein content and are expressed as ng of hK13 per mg of total protein.

FIG. 5 show the results of the quantification of hK13 protein in ovarian tissue extracts. Extracts from 10 normal ovaries were analyzed, 10 tissues with benign disease and 20 with ovarian cancer. Results are expressed as ng of hK13 per mg of total protein. In 50% of the ovarian cancer tissue extracts, hK13 protein is overexpressed, in comparison to normal and benign tissue extracts.

FIG. 6 shows the results of the fractionation of ascites fluid from an ovarian cancer patient by size exclusion high-performance liquid chromatography. The fractions were analyzed for hK13 by the developed ELISA assay. There are two immunoreactive peaks, one at fractions 38-39, corresponding to a molecular weight of approximately 30 kDa (free hK13). Another, smaller peak elutes at fractions 30-32 and corresponds to a molecular weight of approximately 100 kDa. This peak may represent hK13 bound to a proteinase inhibitor.

FIG. 7 shows the results of immunohistochemical localization of hK13 protein in paraffin-embedded prostatic tissues (needle biopsies) using the polyclonal hK13 rabbit antibody. There is moderate cytoplasmic positivity with minimal nuclear staining and negative stroma. (A). Prostate cancer, case 1. (B). Prostate hyperplasia, case 1. (C). Prostate cancel, case 2. (D). Prostate cancer and hyperplasia, case 3. (Original magnifications A-D×400).

FIG. 8 shows Kaplan-Meier survival curves for A) progression-free survival and B) overall survival in patients with hK13-positive and negative ovarian tumors. n=number of samples

### DETAILED DESCRIPTION OF THE INVENTION

The invention relates to newly discovered correlations between expression of kallikrein 13 and endocrine cancer. The methods described herein provide sensitive methods for detecting endocrine cancer. The level of expression of kallikrein 13 correlates with the presence or absence of endocrine cancer. Methods are provided for detecting the presence of endocrine cancer in a sample, the absence of endocrine cancer in a sample, assessing the histology of tissues associated with endocrine cancer, and other characteristics of endocrine cancer that are relevant to prevention, diagnosis, characterization, and therapy of endocrine cancer in a patient. In particular, the invention provides methods for assessing prognosis of a patient with ovarian cancer. Methods are also provided for assessing the efficacy of one or more test agents for modulating kallikrein 13 that affect endocrine cancer, assessing the efficacy of a therapy for endocrine cancer, monitoring the progression of endocrine cancer, selecting an agent or therapy for inhibiting endocrine cancer, treating a patient afflicted with endocrine cancer, inhibiting endocrine cancer in a patient, and assessing the potential of a test compound to contribute to endocrine cancer.

Glossary

Samples that may be analyzed using the methods of the invention include those that are known or suspected to express kallikrein 13 or contain kallikrein 13. The terms “sample”, “biological sample”, and the like mean a material known or suspected of expressing or containing kallikrein 13. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, cytosolic extracts, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins may be isolated from the samples and utilized in the methods of the invention.

“Endocrine cancer” or “endocrine carcinoma” includes but is not limited to cancers of reproductive organs such as ovarian cancer and breast cancer, and prostate cancer. In particular aspects of the invention, the endocrine cancer is ovarian cancer.

The term “subject”, “individual”, or “patient” refers to a warm-blooded animal such as a mammal which is afflicted with endocrine cancer or condition as described herein. In particular, the terms refer to a human.

The term “kallikrein 13” includes human kallikrein 13 (“hK13”) and in particular the native-sequence polypeptide, all homologs, isoforms, fragments, chimeric polypeptides, and precursors of human kallikrein 13. The amino acid sequence for native hK13 include the sequences of GenBank Accession Nos. AAG23259, NM015596, AF135024, and NP05641 and of SEQ ID NO. 1).

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to the sequences identified in the GenBank Accession Nos. AAG23259, NM015596, AF135024, and NP056411, and shown in SEQ ID NO. 1. Such variants include, for instance, polypeptides wherein one or more amino acid residues are added to, or deleted from, the N- or C-terminus of the full-length or mature sequences of SEQ ID NO: 1, including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a kallikrein polypeptide homolog, for example, the murine kallikrein polypeptide.

The invention also includes polypeptides that are substantially identical to the sequences of GenBank Accession Nos. AAG23259, NM015596, AF135024, and NP056411 and SEQ ID NO. 1 (e.g. at least about 45%, preferably 50%, 55%, 60%, 65%, 70%, 75%, 80%, 85%, 90%, 95%, 97%, 98%, or 99% sequence identity), and in particular polypeptides that retain the immunogenic activity of the corresponding native-sequence polypeptide.

Percent identity of two amino acid sequences, or of two nucleic acid sequences identified herein is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403 410, 1990). Skilled artisans can determine appropriate parameters for measuring alignment, including any algorithms needed to achieve maximal alignment over the full length of the sequences being compared. Methods to determine identity and similarity are codified in publicly available computer programs.

Kallikrein 13 polypeptides include chimeric or fusion proteins. A “chimeric protein” or “fusion protein” comprises all or part (preferably biologically active) of a polypeptide operably linked to a heterologous polypeptide (i.e., a polypeptide other than a kallikrein 13 polypeptide). Within the fusion protein, the term “operably linked” is intended to indicate that a kallikrein 13 polypeptide and the heterologous polypeptide are fused in-frame to each other. The heterologous polypeptide can be fused to the N-terminus or C-terminus of a kallikrein 13 polypeptide. A useful fusion protein is a GST fusion protein in which a kallikrein 13 polypeptide is fused to the C-terminus of GST sequences. Another example of a fusion protein is an immunoglobulin fusion protein in which all or part of a kallikrein 13 polypeptide is fused to sequences derived from a member of the immunoglobulin protein family. Chimeric and fusion proteins can be produced by standard recombinant DNA techniques.

Kallikrein polypeptides may be isolated from a variety of sources, such as from human tissue types or from another source, or prepared by recombinant or synthetic methods, or by any combination of these and similar techniques.

The term “KLK13” or “KLK13 nucleic acid(s)” are intended to include DNA and RNA (e.g. mRNA) and can be either double stranded or single stranded. The term includes nucleic acids that encode a native-sequence polypeptide, a polypeptide variant including a portion of a kallikrein polypeptide, an isoform, precursor, and chimeric polypeptide. The nucleic acid sequences encoding native kallikrein polypeptides employed in the present invention include the nucleic acid sequences of GenBank Accession No. AF135024 and in SEQ ID NO: 2, or fragments thereof

“Statistically different levels” or “significant differences” in levels of markers in a patient sample compared to a control or standard (e.g. normal levels or levels in other samples from a patient) may represent levels that are higher or lower than the standard error of the detection assay. The levels may be at least 1.5, 2, 3, 4, 5, or 6 times higher or lower than the control or standard.

“Binding agent” refers to a substance such as a polypeptide or antibody that specifically binds to a kallikrein 13. A substance “specifically binds” to a kallikrein 13 if it reacts at a detectable level with a kallikrein 13, and does not react detectably with peptides containing unrelated sequences or sequences of different polypeptides. Binding properties may be assessed using an ELISA, which may be readily performed by those skilled in the art (see for example, Newton et al, Develop. Dynamics 197: 1-13, 1993).

A binding agent may be a ribosome, with or without a peptide component, an RNA molecule, or a polypeptide. A binding agent may be a polypeptide that comprises a kallikrein 13 polypeptide sequence, a peptide variant thereof, or a non-peptide mimetic of such a sequence. By way of example a kallikrein 13 sequence may be a peptide portion of a kallikrein 13 that is capable of modulating a function mediated by the kallikrein 13.

Methods of Detecting Kallikrein 13

A variety of methods can be employed for the diagnostic and prognostic evaluation of endocrine cancer involving kallikrein 13, and the identification of subjects with a predisposition to such disorders. Such methods may, for example, utilize antibodies directed against kallikrein 13, including peptide fragments. In particular, antibodies may be used, for example, for the detection of either an over- or an under-abundance of kallikrein 13 relative to a non-disorder state or the presence of a modified (e.g., less than full length) kallikrein 13 which correlates with a disorder state, or a progression toward a disorder state.

The invention also contemplates a method for detecting endocrine cancer comprising producing a profile of levels of kallikrein 13 and other markers associated with endocrine cancer in cells from a patient, and comparing the profile with a reference to identify a profile for the test cells indicative of disease.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. For example, the presence of kallikrein 13 may be indicative of a favorable prognosis. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring endocrine carcinoma by detecting kallikrein 13 in biological samples from a subject. These applications require that the amount of kallikrein 13 quantitated in a sample from a subject being tested be compared to levels quantitated for another sample or an earlier sample from the subject, or levels quantitated for a control sample. Levels for control samples from healthy subjects or endocrine cancer subjects may be established by prospective and/or retrospective statistical studies. Healthy subjects who have no clinically evident disease or abnormalities may be selected for statistical studies. Diagnosis may be made by a finding of statistically different levels of kallikrein 13 compared to a control sample or previous levels quantitated for the same subject.

Binding agents specific for kallikrein 13 may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of endocrine cancer in a subject may be determined by (a) contacting a sample from the subject with a binding agent for kallikrein 13; (b) detecting in the sample levels of kallikrein 13 that bind to the binding agent; and (c) comparing the levels of kallikrein 13 with a predetermined standard or cut-off value.

In particular embodiments, the binding agent is an antibody.

In an aspect, the invention provides a diagnostic method for monitoring or diagnosing endocrine cancer in a subject by quantitating kallikrein 13 in a biological sample from the subject comprising reacting the sample with antibodies specific for kallikrein 13, which are directly or indirectly labeled with a detectable substance and detecting the detectable substance.

In an aspect of the invention, a method for detecting endocrine cancer is provided comprising:
(a) obtaining a sample suspected of containing kallikrein 13 associated with endocrine cancer;
(b) contacting said sample with antibodies that specifically bind kallikrein 13 under conditions effective to bind the antibodies and form complexes;
(c) measuring the amount of kallikrein 13 present in the sample by quantitating the amount of the complexes; and
(d) comparing the amount of kallikrein 13 present in the samples with the amount of kallikrein 13 in a control, wherein a change or significant difference in the amount of kallikrein 13 in the sample compared with the amount in the control is indicative of endocrine cancer.

In an embodiment, the invention contemplates a method for monitoring the progression of endocrine cancer in an individual, comprising:
(a) contacting an amount of an antibody which binds to a kallikrein 13 protein, with a sample from the individual so as to form a complex comprising the antibody and kallikrein 13 protein in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the cancer in said individual.

In an embodiment, the invention contemplates a method for monitoring the progression of endocrine cancer in an individual, comprising:
(a) contacting antibodies which bind to kallikrein 13 with a sample from the individual so as to form binary complexes comprising the antibodies and kallikrein 13 in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the stage and/or progression of the endocrine cancer in said individual.

The amount of complexes may also be compared to a value representative of the amount of the complexes from an individual not at risk of, or afflicted with, endocrine cancer at different stages. A significant difference in complex formation may also be indicative of advanced disease e.g. advanced endocrine cancer. A significant difference in complex formation may also be indicative of a favorable prognosis.

In a particular embodiment, a method is provided for determining the prognosis of an individual with ovarian cancer, in particular an individual with non-serous epithelial tumors, comprising:
(a) contacting an amount of an antibody which binds to a kallikrein 13 protein, with a sample from the patient so as to form a complex comprising the antibody and kallikrein 13 protein in the sample;
(b) determining or detecting the presence or amount of complex formation in the sample;
(c) comparing the amount of kallikrein 13 present in the sample with the amount of kallikrein 13 in a control, wherein a higher amount of kallikrein 13 in the sample compared with the amount in the control is indicative of a favourable prognosis, in particular, early stage disease, no residual tumor, optimal debulking success, longer PFS and/or OS.

In an embodiment of a method for determining the prognosis of an individual with ovarian cancer, the sample is contacted with antibodies which bind to kallikrein 13 and one or both of kallikrein 5 and kallikrein 11.

Antibodies specifically reactive with a kallikrein 13 protein, or derivatives, such as enzyme conjugates or labeled derivatives, may be used to detect kallikrein 13 protein in various samples (e.g. biological materials). They may be used as diagnostic or prognostic reagents and they may be used to detect abnormalities in the level of kallikrein 13 expression, or abnormalities in the structure, and/or temporal, tissue, cellular, or subcellular location of kallikrein 13. Antibodies may also be used to screen potentially therapeutic compounds in vitro to determine their effects on disorders (e.g. endocrine cancer) involving a kallikrein 13 protein, and other conditions. In vitro immunoassays may also be used to assess or monitor the efficacy of particular therapies.

Antibodies may be used in any known immunoassays that rely on the binding interaction between an antigenic determinant of a kallikrein 13 protein and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify kallikrein 13 in a sample in order to diagnose and treat pathological states.

In particular, the antibodies may be used in immunohistochemical analyses, for example, at the cellular and sub-subcellular level, to detect a kallikrein 13 protein, to localize it to particular endocrine tumor cells and tissues, and to specific subcellular locations, and to quantitate the level of expression.

Antibodies for use in the present invention include but are not limited to monoclonal or polyclonal antibodies, immunologically active fragments (e.g. a Fab or (Fab)₂ fragment), antibody heavy chains, humanized antibodies, antibody light chains, genetically engineered single chain Fᵥ molecule (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labelled derivatives.

Antibodies including monoclonal and polyclonal antibodies, fragments and chimeras, nay be prepared using methods known to those skilled in the art (See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies.) Antibodies specific for kallikrein 13 may also be obtained from scientific or commercial sources.

In an embodiment of the invention, antibodies are reactive against kallikrein 13 if they bind with a Kₐ of greater than or equal to 10⁻⁷ M.

In a sandwich immunoassay of the invention mouse polyclonal/monoclonal antibodies against kallikrein 13 and rabbit polyclonal/monoclonal antibodies against kallikrein 13 may be utilized.

An antibody specific for kallikrein 13 may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which a primary antigen-antibody reaction is amplified by the introduction of a second antibody, having specificity for the antibody reactive against kallikrein 13. By way of example, if the antibody having specificity against kallikrein 13 is a rabbit IgG antibody, the second antibody may be goat anti-rabbit IgG, Fc fragment specific antibody labelled with a detectable substance as described herein.

Methods for conjugating or labelling the antibodies discussed above may be readily accomplished by one of ordinary skill in the art (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect a kallikrein 13 protein. Generally, an antibody may be labeled with a detectable substance and a kallikrein 13 protein may be localised in tissues and cells based upon the presence of the detectable substance.

In the context of the methods of the invention, the sample, binding agents (e.g. antibodies) or kallikrein 13 may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for kallikrein 13 may be immobilized using sheep anti-mouse IgG Fc fragment specific antibody coated on the carrier or support.

Where a radioactive label is used as a detectable substance, a kallikrein 13 protein may be localized by radioautography. The results of radioautography may be quantitated by determining the density of particles in the radioautographs by various optical methods, or by counting the grains.

Time-resolved fluorometry may be used to detect a fluorescent signal, label, or detectable substance. For example, the method described in Christopoulos T K and Diamandis E P Anal. Chem., 1992:64:342-346 may be used with a conventional time-resolved fluorometer.

Therefore, in accordance with an embodiment of the invention, a method is provided wherein a kallikrein 13 antibody is directly or indirectly labelled with an enzyme, a substrate for the enzyme is added wherein the substrate is selected so that the substrate, or a reaction product of the enzyme and substrate, forms fluorescent complexes with a lanthanide metal (e.g. europium, terbium, samarium, and dysprosium, preferably europium and terbium). A lanthanide metal is added and kallikrein 13 is quantitated in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,312,922 to Diamandis. Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

Antibodies specific for kallikrein 13 may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein. In an embodiment, an antibody specific for the anti-kallikrein 13 antibody is labelled with an enzyme.

In accordance with an embodiment, the present invention provides means for determining kallikrein 13 in a sample by measuring kallikrein 13 by immunoassay. It will be evident to a skilled artisan that a variety of immunoassay methods can be used to measure kallikrein 13 in a sample. In general, a kallikrein 13 immunoassay method may be competitive or noncompetitive. Competitive methods typically employ an immobilized or immobilizable antibody to kallikrein 13 (anti-kallikrein 13) and a labeled form of kallikrein 13. Sample kallikrein 13 and labeled kallikrein 13 compete for binding to anti-kallikrein 13. After separation of the resulting labeled kallikrein 13 that has become bound to anti-kallikrein 13 (bound fraction) from that which has remained unbound (unbound fraction), the amount of the label in either bound or unbound fraction is measured and may be correlated with the amount of kallikrein 13 in the test sample in any conventional manner, e.g., by comparison to a standard curve.

In an aspect, a non-competitive method is used for the determination of kallikrein 13, with the most common method being the “sandwich” method. In this assay, two anti-kallikrein 13 antibodies are employed. One of the anti-kallikrein 13 antibodies is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody may be separated from the liquid test mixture, and the label may be measured in at least a portion of the separated capture antibody phase or the remainder of the liquid test mixture. Generally it is measured in the capture antibody phase since it comprises kallikrein 13 bound by (“sandwiched” between) the capture and detection antibodies. In another embodiment, the label may be measured without separating the capture antibody and liquid test mixture.

In a typical two-site immunometric assay for kallikrein 13, one or both of the capture and detection antibodies are polyclonal antibodies or one or both of the capture and detection antibodies are monoclonal antibodies (e.g. polyclonal/polyclonal assay, monoclonal/monoclonal assay, or monoclonal/polyclonal assay). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. In an embodiment, the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

A particular sandwich immunoassay method of the invention employs two antibodies reactive against kallikrein 13, a second antibody having specificity against an antibody reactive against kallikrein 13 labelled with an enzymatic label, and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(³⁺)-EDTA, which can be quantified by measuring the Tb³⁺ fluorescence in a time-resolved mode. Fluorescence intensity is measured using a time-resolved fluorometer as described herein.

The above-described immunoassay methods and formats are intended to be exemplary and are not limiting.

The invention also contemplates the methods described herein using multiple markers for endocrine cancer. Therefore, the invention contemplates a method for anaylzing a biological sample for the presence of kallikrein 13 and other markers that are specific indicators of endocrine cancer. Other markers include human stratum corneum chymotryptic enzyme (HSCCE), haptoglobin alpha, osteopontin, kallikrein 2, kallikrein 3, kallikrein 4, kallikrein 5, kallikrein 6, kallikrein 7, kallikrein 8, kallikrein 9, kallikrein 10, kallikrein 11, kallikrein 14, and kallikrein 15; CA125, CA15-3, CA19-9, OVX1, lysophosphatidic acid (LPA), creatin-kinase BB, and carcinoembryonic antigen (CEA). Preferably the other markers are markers to kallikreins. In an aspect of the invention, the markers are one or more of hK4, hK5, hK6, hK7, hK8, hK9, hK10, hK11, hK14, and hK15, in particular hK4, hK5, hK6, hK7, hK10, and hK15 or hK8, hK9, hK11, and hK14, more particularly hK6, hK10, and hK11. The methods described herein may be modified by including reagents to detect the markers, or nucleic acids for the markers. The methods described herein may also include reagents to detect KLK13. Techniques for detecting nucleic acid such as polymerase chain reaction (PCR) and hybridization assays are well known in the art.

Computer Systems

Computer readable media comprising kallikrein 13 and optionally other markers of endocrine cancer is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on kallikrein 13 and optionally other endocrine cancer markers.

A variety of data processor programs and formats can be used to store information on kallikrein 13 and other endocrine cancer markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has endocrine cancer or a pre-disposition to endocrine cancer, comprising determining the presence or absence of kallikrein 13 and optionally other endocrine cancer markers, and based on the presence or absence of the kallikrein 13 and optionally other markers, determining whether the patient has endocrine cancer or a pre-disposition to endocrine cancer, and optionally recommending treatment for the endocrine cancer or pre-disease condition.

The invention also provides in an electronic system and/or in a network, a method for determining whether a subject has endocrine cancer or a pre-disposition to endocrine cancer, comprising determining the presence or absence of kallikrein 13 and optionally other endocrine cancer markers, and based on the presence or absence of the kallikrein 13 and optionally other markers, determining whether the subject has endocrine cancer or a pre-disposition to endocrine cancer, and optionally recommending treatment for endocrine cancer or pre-disease condition.

The invention further provides in a network, a method for determining whether a subject has endocrine cancer or a pre-disposition to endocrine cancer comprising: (a) receiving phenotypic information on the subject and information on kallikrein 13 and optionally other endocrine cancer markers associated with samples from the subject; (b) acquiring information from the network corresponding to the kallikrein 13 and optionally other markers; and (c) based on the phenotypic information and information on the kallikrein 13 and optionally other markers determining whether the subject has endocrine cancer or a pre-disposition to endocrine cancer; and (d) optionally recommending treatment for the endocrine cancer or pre-disease condition.

The invention still further provides a system for identifying selected records that identify an endocrine cancer cell or tissue. A system of the invention generally comprises a digital computer; a database server coupled to the computer; a database coupled to the database server having data stored therein, the data comprising records of data comprising kallikrein 13 and optionally other endocrine cancer markers, or nucleic acids encoding same, and a code mechanism for applying queries based upon a desired selection criteria to the data file in the database to produce reports of records which match the desired selection criteria.

In an aspect of the invention a method is provided for detecting endocrine cancer tissue or cells using a computer having a processor, memory, display, and input/output devices, the method comprising the steps of:
(a) creating records of kallikrein 13 and optionally other endocrine cancer markers isolated from a sample suspected of containing endocrine cancer cells or tissue;
(b) providing a database comprising records of data comprising kallikrein 13 and optionally other endocrine cancer markers; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are endocrine cancer cells or tissue.

The invention contemplates a business method for determining whether a subject has endocrine cancer or a pre-disposition to endocrine cancer comprising: (a) receiving phenotypic information on the subject and information on kallikrein 13 and optionally other endocrine cancer markers associated with samples from the subject; (b) acquiring information from a network corresponding to kallikrein 13 and optionally other markers; and (c) based on the phenotypic information, information on kallikrein 13 and optionally other markers, and acquired information, determining whether the subject has endocrine cancer or a pre-disposition to endocrine cancer; and (d) optionally recommending treatment for the endocrine cancer or pre-condition.

In an aspect, the computer systems, components, and methods described herein may be used to monitor or assess the prognosis of an individual with ovarian cancer.

Imaging Methods

Antibodies specific for kallikrein 13 may also be used in imaging methodologies in the management of endocrine cancer. The invention provides a method for imaging tumors associated with one or more kallikreins, preferably kallikreins associated with endocrine cancer, most preferably kallikrein 13 and optionally one or more kallikreins specifically selected from hK2, hK3, hK4, hK5, hK6, hK7, hK5, hK9, hK10, hK11, hK14, and hK15.

In an embodiment, a method of the invention comprises administering to a tissue of a subject with endocrine cancer imaging agents that carry imagining labels and are capable of targeting or binding to kallikrein 13 and optionally other endocrine cancer markers. The agent is allowed to incubate and bind to the kallikrein 13 and optionally other markers.

In another embodiment the method is an in vivo method and a subject or patient is administered one or more agents that carry an imaging label and are capable of targeting or binding to kallikrein 13 and optionally other markers of endocrine cancer. The imaging agent is allowed to incubate in vivo and bind to kallikrein 13 and optionally other markers of endocrine cancer associated with a tumor, preferably endocrine tumors. The presence of the label is localized to the endocrine cancer, and the localized label is detected using imaging devices known to those skilled in the art.

The invention also contemplates imaging methods described herein using multiple markers for endocrine cancer. For example, a method for imaging endocrine cancer may further comprise injecting the patient with one or more of an agent that binds to human stratum corneum chymotryptic enzyme (HSCCE), haptoglobin alpha, kallikrein 2, kallikrein 3, kallikrein 4, kallikrein 5, kallikrein 6, kallikrein 7, kallikrein 8, kallikrein 9, kallikrein 10, kallikrein 11, kallikrein 14, kallikrein 15, CA125, CA15-3, CA19-9, OVX1, lysophosphatidic acid (LPA), carcinoembryonic antigen (CEA), prostate specific antigen, prostate specific membrane antigen, or prostate stem cell antigen. Preferably each agent is labeled so that it can be distinguished during the imaging.

The imaging agent may be an antibody or chemical entity that recognizes kallikrein 13 and optionally other markers of endocrine cancer. In an aspect of the invention the agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for kallikrein 13 and optionally other markers of endocrine cancer used in the methods of the invention may be obtained from scientific or commercial sources, or isolated native protein or recombinant protein may be utilized to prepare antibodies etc. as described herein.

An imaging agent may be a peptide that mimics the epitope for an antibody specific for a marker (e.g. Kallikrein 13) and binds to the marker. The peptide may be produced on a commercial synthesizer using conventional solid phase chemistry. By way of example, a peptide may be prepared that includes either tyrosine lysine, or phenylalanine to which N₂S₂ chelate is complexed (See U.S. Pat. No. 4,897,255). The anti-kallikrein peptide conjugate is then combined with a radiolabel (e.g. sodium ^{99m}-Tc pertechnetate or sodium ¹⁸⁸Re perrhenate) and it may be used to locate a kallikrein producing tumor.

An imaging agent carries a label to image kallikrein 13 and optionally other markers. The imaging agent may be labelled for use in radionuclide imaging. In particular, the agent may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, 3H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ¹⁹¹m-¹⁹¹Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m}Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An agent may also be labeled with a paramagnetic isotope for purposes of an in vivo method of the invention. Examples of elements that are useful in magnetic resonance imaging include gadolinium, terbium, tin, iron, or isotopes thereof (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

In the case of a radiolabeled agent, the agent may be administered to the patient, it is localized to the tumor having a kallikrein with which the agent binds, and is detected or “imaged” in vivo using known techniques such as radionuclear scanning using e.g., a gamma camera or emission tomography. [See for example A. R. Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for kallikreins, or fragments thereof having the same epitope specificity, are bound to a suitable radioisotope, or a combination thereof, and administered parenterally. The bio-distribution of the label can be monitored by scintigraphy, and accumulations of the label are related to the presence of endocrine cancer cells. Whole body imaging techniques are described in U.S. Pat. Nos. 4,036,945 and 4,311,688. Other examples of agents useful for diagnosis and therapeutic use that can be coupled to antibodies and antibody fragments include metallothionein and fragments (see, U.S. Pat. No. 4,732,864). These agents are useful in diagnosis staging and visualization of cancer, in particular endocrine cancer, so that surgical and/or radiation treatment protocols can be used more efficiently.

An imaging agent may carry a bioluminescent or chemiluminescent label. Such labels include polypeptides known to be fluorescent, bioluminescent or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be utilized in a method of the invention. For example, the chemiluminescent polypeptide can be luciferase and the reagent luciferin. A substrate for a bioluminescent or chemiluminescent label can be administered before, at the same time (e.g., in the same formulation), or after administration of the agent.

An imaging agent may comprise a paramagnetic compound, such as a polypeptide chelated to a metal, e.g.; a metalloporphyrin. The paramagnetic compound may also comprise a monocrystalline nanoparticle, e.g., a nanoparticle comprising a lanthanide (e.g., Gd) or iron oxide; or, a metal ion comprising a lanthanide. “Lanthanides” refers to elements of atomic numbers 58 to 70, a transition metal of atomic numbers 21 to 29, 42 or 44, a Gd(II), a Mn(II), or an element comprising an Fe element. Paramagnetic compounds can also comprise a neodymium iron oxide (NdFeO.sub.3) or a dysprosium iron oxide (DyFeO.sub.3). Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

An image can be generated in a method of the invention by computer assisted tomography (CAT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be utilized in the practice of the present invention. (See, for example, U.S. Pat. Nos. 6,151,377; 5,946,371; 5,446,799; 5,406,479; 5,208,581; 5,109,397). The invention may also utilize animal imaging modalities, such as MicroCAT.™. (ImTek, Inc.).

Magnetic resonance imaging (MRI) systems and devices well known in the art can be utilized in the practice of the present invention. In magnetic resonance methods and devices, a static magnetic field is applied to a tissue or a body in order to define an equilibrium axis of magnetic alignment in a region of interest. A radio frequency field is then applied to the region in a direction orthogonal to the static magnetic field direction to excite magnetic resonance in the region. The resulting radio frequency signals are then detected and processed, and the exciting radio frequency field is applied. The resulting signals are detected by radio-frequency coils that are placed adjacent to the tissue or area of the body of interest. (For a description of MRI methods and devices see, for example, U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,378,987; 5,214,382; 5,031,624; 5,207,222; 4,985,678; 4,906,931; 4,558,279). MRI and supporting devices are commercially available for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. The invention may also utilize animal imaging modalities such as micro-MRIs.

Positron emission tomography imaging (PET) systems and devices well known in the art can be utilized in the practice of the present invention. For example, a method of the invention may use the system designated Pet VI located at Brookhaven National Laboratory. For descriptions of PET systems and devices see, for example, U.S. Pat. Nos. 6,151,377; 6,072,177; 5,900,636; 5,608,221; 5,532,489; 5,272,343; 5,103,098. Animal imaging modalities such as micro-PETs (Corcorde Microsystems, Inc.) can also be used in the invention.

Single-photon emission computed tomography (SPECT) systems and devices well known in the art can be utilized in the practice of the present invention. (See, for example, U.S. Pat. Nos. 6,115,446; 6,072,177; 5,608,221; 5,600,145; 5,210,421; 5,103,098.) The methods of the invention may also utilize animal imaging modalities, such as micro-SPECTs.

Bioluminescence imaging includes bioluminescence, fluorescence or chemiluminescence or other photon detection systems and devices that are capable of detecting bioluminescence, fluorescence or chemiluminescence. Sensitive photon detection systems can be used to detect bioluminescent and fluorescent proteins externally; see, for example, Contag (2000) Neoplasia 2:41-52; Zhang (1994) Clin. Exp. Metastasis 12:87-92. The methods of the invention can be practiced using any such photon detection device, or variation or equivalent thereof, or in conjunction with any known photon detection methodology, including visual imaging. By way of example, an intensified charge-coupled device (ICCD) camera coupled to an image processor may be used in the present invention. (See, e.g., U.S. Pat. No. 5,650,135). Photon detection devices are also commercially available from Xenogen, Hamamatsue.

Screening Methods

The invention also contemplates methods for evaluating test agents or compounds for their ability to inhibit endocrine cancer or potentially contribute to endocrine cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting endocrine cancer in a patient, the method comprising comparing:
(a) levels of kallikrein 13 and optionally other endocrine cancer markers in a first sample obtained from a patient and exposed to the test agent; and
(b) levels of kallikrein 13 and optionally other markers in a second sample obtained from the patient, wherein the sample is not exposed to the test agent, wherein a significant difference in the levels of expression of kallikrein 13 and optionally the other markers in the first sample, relative to the second sample, is an indication that the test agent is potentially efficacious for inhibiting endocrine cancer in the patient.

The first and second samples may be portions of a single sample obtained from a patient or portions of pooled samples obtained from a patient.

In an embodiment, the levels of expression of hK5 in the first sample are significantly lower relative to the second sample. In an embodiment, the levels of expression of hK5 in the first sample are significantly higher relative to the second sample.

In an aspect, the invention provides a method of selecting an agent for inhibiting endocrine cancer in a patient comprising:
(a) obtaining a sample from the patient;
(b) separately maintaining aliquots of the sample in the presence of a plurality of test agents;
(c) comparing kallikrein 13 and optionally other endocrine cancer markers, in each of the aliquots; and
(d) selecting one of the test agents which alters the levels of kallikrein 13 and optionally other endocrine cancer markers in the aliquot containing that test agent, relative to other test agents.

In an embodiment, the levels of kallikrein 13 are significantly lower in the presence of the selected test agent. In an embodiment, the levels of kallikrein 13 are significantly higher in the presence of the selected test agent.

Still another aspect of the present invention provides a method of conducting a drug discovery business comprising:
(a) providing one or more methods or assay systems for identifying agents that inhibit endocrine cancer in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to endocrine cancer comprising:
(a) maintaining separate aliquots of cells or tissues from a patient with endocrine cancer in the presence and absence of the test compound; and
(b) comparing kallikrein 13 and optionally other endocrine cancer markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to endocrine cancer. In an embodiment, the levels of kallikrein 13 are lower in the presence of the test compound. In another embodiment, the levels of kallikrein 13 are higher in the presence of the test compound.

Kits

The invention also contemplates kits for carrying out the methods of the invention. Such kits typically comprise two or more components required for performing a diagnostic assay. Components include but are not limited to compounds, reagents, containers, and/or equipment

The methods described herein may be performed by utilizing pre-packaged diagnostic kits comprising at least binding agents (e.g. antibodies) described herein, which may be conveniently used, e.g., in clinical settings, to screen and diagnose patients, and to screen and identify those individuals afflicted with or exhibiting a predisposition to endocrine cancer.

In an embodiment, a container with a kit comprises binding agents as described herein. By way of example, the kit may contain antibodies specific for kallikrein 13 and optionally other endocrine cancer markers, antibodies against the antibodies labelled with enzymes, and substrates for the enzymes. The kit may also contain microtiter plate wells, standards, assay diluent, wash buffer, adhesive plate covers, and/or instructions for carrying out a method of the invention using the kit.

In an aspect, a kit includes an antibody or an antibody fragment which binds specifically to an epitope of kallikrein 13, and means for detecting binding of the antibody to its epitope associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains a radiolabelled antibody preparation for in vivo imaging.

The reagents suitable for applying the screening methods of the invention to evaluate compounds may be packaged into convenient kits described herein providing the necessary materials packaged into suitable containers.

Thus, the invention relates to a kit for assessing the suitability of each of a plurality of test compounds for inhibiting endocrine cancer in a patient. The kit comprises reagents for assessing kallikrein 13 and optionally a plurality of test agents or compounds.

The invention contemplates a kit for assessing the presence of endocrine cancer cells, wherein the kit comprises antibodies specific for kallikrein 13, and optionally antibodies specific for other markers associated with endocrine cancer.

Additionally the invention provides a kit for assessing the potential of a test compound to contribute to endocrine cancer. The kit comprises endocrine cancer cells and reagents for assessing kallikrein 13, and optionally other markers associated with endocrine cancer.

Therapeutic Applications

Since kallikrein 13 is expressed or overexpressed in endocrine cancers, such as ovarian and prostate cancer, it is a target for cancer immunotherapy. Such immunotherapeutic methods include the use of antibody therapy, in vivo vaccines, and ex viva immunotherapy approaches.

In one aspect, the invention provides kallikrein 13 antibodies that may be used systemically to treat cancer, such as ovarian and prostate cancer. Preferably antibodies are used that target the tumor cells but not the surrounding non-tumor cells and tissue. Thus, the invention provides a method of treating a patient susceptible to, or having a cancer that expresses kallikrein 13, comprising administering to the patient an effective amount of an antibody which binds specifically to kallikrein 13. In another aspect, the invention provides a method of inhibiting the growth of tumor cells expressing kallikrein 13, comprising administering to a patient an antibody which binds specifically to kallikrein 13 in an amount effective to inhibit growth of the tumor cells. Kallikrein 13 antibodies may also be used in a method for selectively inhibiting the growth of or killing a cell expressing kallikrein 13 comprising reacting a kallikrein 13 antibody immunoconjugate or immunotoxin with the cell in an amount sufficient to inhibit the growth of or kill the cell.

By way of example, unconjugated kallikrein 13 antibody may be introduced into a patient such that the antibody binds to kallikrein 13 expressing cancer cells and mediates growth inhibition of such cells (including the destruction thereof), and the tumor, by mechanisms which may include complement-mediated cytolysis, antibody-dependent cellular cytotoxicity, altering the physiologic function of kallikrein 13, and/or the inhibition of ligand binding or signal transduction pathways. In addition to unconjugated kallikrein 13 antibodies, kallikrein 13 antibodies conjugated to therapeutic agents (e.g. immunoconjugates) may also be used therapeutically to deliver the agent directly to kallikrein 13 expressing tumor cells and thereby destroy the tumor. Examples of such agents include abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Cancer immunotherapy using kallikrein 13 antibodies may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J Clin Immunol 11: 117-127).

In the practice of the method of the invention, anti-kallikrein 13 antibodies capable of inhibiting the growth of cancer cells expressing kallikrein 13 are administered in a therapeutically effective amount to cancer patients whose tumors express or overexpress kallikrein 13. The invention may provide a specific, effective and long-needed treatment for endocrine cancers including ovarian or prostate cancer. The antibody therapy methods of the invention may be combined with other therapies including chemotherapy and radiation.

Patients may be evaluated for the presence and level of kallikrein 13 expression and overexpression in tumors, preferably using immunohistochemical assessments of tumor tissue, quantitative kallikrein 13 imaging as described herein, or other techniques capable of reliably indicating the presence and degree of kallikrein 13 expression. Immunohistochemical analysis of tumor biopsies or surgical specimens may be employed for this purpose.

Anti-kallikrein 13 antibodies useful in treating cancer include those that are capable of initiating a potent immune response against the tumor and those that are capable of direct cytotoxicity. In this regard, anti-kallikrein 13 antibodies may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, anti-kallikrein 13 antibodies which exert a direct biological effect on tumor growth are useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-kallikrein 13 antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a particular anti-kallikrein 13 antibody, or combination of anti-kallikrein 13 antibodies, may be evaluated in vivo using a suitable animal model. Xenogenic cancer models, wherein human cancer explants or passaged xenograft tissues are introduced into immune compromised animals, such as nude or SCID mice, may be employed.

The methods of the invention contemplate the administration of single anti-kallikrein 13 antibodies as well as combinations, or “cocktails”, of different individual antibodies such as those recognizing different epitopes or other kallikreins. Such cocktails may have certain advantages inasmuch as they contain antibodies which bind to different epitopes or kallikreins and/or exploit different effector mechanisms or combine directly cytotoxic antibodies with antibodies that rely on immune effector functionality. Such antibodies in combination may exhibit synergistic therapeutic effects. In addition, the administration of anti-kallikrein 13 antibodies may be combined with other therapeutic agents, including but not limited to chemotherapeutic agents, androgen-blockers, and immune modulators (e.g., IL2, GM-CSF). The anti-kallikrein 13 antibodies may be administered in their “naked” or unconjugated form, or may have therapeutic agents conjugated to them.

The anti-kallikrein 13 antibodies used in the practice of the method of the invention may be formulated into pharmaceutical compositions comprising a carrier suitable for the desired delivery method. Suitable carriers include any material which when combined with the antibodies retains the anti-tumor function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Anti-kallikrein 13 antibody formulations may be administered via any route capable of delivering the antibodies to the tumor site. Routes of administration include, but are not limited to, intravenous, intraperitoneal, intramuscular, intratumor, intradermal, and the like. Preferably, the route of administration is by intravenous injection. Anti-kallikrein 13 antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of cancer and the severity, grade, or stage of the cancer, the binding affinity and half life of the antibodies used, the degree of kallikrein 13 expression in the patient, the extent of circulating kallikrein 13 antigen, the desired steady-state antibody concentration level, frequency of treatment, and the influence of any chemotherapeutic agents used in combination with the treatment method of the invention.

Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve tumor inhibition or regression. Direct administration of kallikrein 13 antibodies is also possible and may have advantages in certain situations.

Patients may be evaluated for serum kallikrein 13 in order to assist in the determination of the most effective dosing regimen and related factors. The kallikrein 13 assay methods described herein, or similar assays, may be used for quantitating circulating kallikrein 13 levels in patients prior to treatment. Such assays may also be used for monitoring throughout therapy, and may be useful to gauge therapeutic success in combination with evaluating other parameters such as serum kallikrein 13 levels.

The invention further provides vaccines formulated to contain a kallikrein 13 protein or fragment thereof. The use in anti-cancer therapy of a tumor antigen in a vaccine for generating humoral and cell-mediated immunity is well known and, for example, has been employed in prostate cancer using human PSMA and rodent PAP immunogens (Hodge et al., 1995, Int. J. Cancer 63: 231-237; Fong et al., 1997, J. Immunol. 159: 3113-3117). These methods can be practiced by employing a kallikrein 13 protein, or fragment thereof, or a kallikrein 13-encoding nucleic acid molecule and recombinant vectors capable of expressing and appropriately presenting the kallikrein 13 immunogen.

By way of example, viral gene delivery systems may be used to deliver a kallikrein 13 encoding nucleic acid molecule. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding a kallikrein 13 protein or fragment thereof introduced into the patient (e.g., intramuscularly) to induce an anti-tumor response.

Various ex vivo strategies may also be employed. One approach involves the use of cells to present kallikrein 13 antigen to a patient's immune system. For example, autologous dendritic cells which express MHC class I and II, may be pulsed with kallikrein 13 or peptides thereof that are capable of binding to MHC molecules, to thereby stimulate cancer (e.g. prostate or ovarian cancer) patients' immune systems (See, for example, Tjoa et al., 1996, Prostate 28: 65-69; Murphy et al., 1996, Prostate 29: 371-380).

Anti-idiotypic anti-kallikrein 13 antibodies can also be used in anti-cancer therapy as a vaccine for inducing an immune response to cells expressing a kallikrein 13 protein. The generation of anti-idiotypic antibodies is well known in the art and can readily be adapted to generate anti-idiotypic anti-kallikrein 13 antibodies that mimic an epitope on a kallikrein 13 protein (see, for example, Wagner et al., 1997, Hybridoma 16: 3340; Foon et al., 1995, J Clin Invest 96: 334-342; Herlyn et al., 1996, Cancer Immunol Immunother 43: 65-76). Such an antibody can be used in anti-idiotypic therapy as presently practiced with other anti-idiotypic antibodies directed against tumor antigens.

Genetic immunization methods may be utilized to generate prophylactic or therapeutic humoral and cellular immune responses directed against cancer cells expressing kallikrein 13. Using the kallikrein 13 encoding DNA molecules, constructs comprising DNA encoding a kallikrein 13 protein/immunogen and appropriate regulatory sequences may be injected directly into muscle or skin of an individual, such that the cells of the muscle or skin take-up the construct and express the encoded kallikrein 13 protein/immunogen. The kallikrein 13 protein/immunogen may be expressed as a cell surface protein or be secreted. Expression of the kallikrein 13 protein/immunogen results in the generation of prophylactic or therapeutic humoral and cellular immunity against the cancer. Various prophylactic and therapeutic genetic immunization techniques known in the art may be used.

The invention further provides methods for inhibiting cellular activity (e.g., cell proliferation, activation, or propagation) of a cell expressing kallikrein 13. This method comprises reacting immunoconjugates of the invention (e.g., a heterogeneous or homogenous mixture) with the cell so that the kallikrein 13 proteins form a complex with the immunoconjugates. A subject with a neoplastic or preneoplastic condition can be treated when the inhibition of cellular activity results in cell death.

In another aspect, the invention provides methods for selectively inhibiting a cell expressing kallikrein 13 by reacting any one or a combination of the immunoconjugates of the invention with the cell in an amount sufficient to inhibit the cell. Amounts include those that are sufficient to kill the cell or sufficient to inhibit cell growth or proliferation.

Kallikrein 13 and fragments thereof, and agents identified using a method of the invention may be used in the treatment of endocrine cancer in a subject. These polypeptides and agents may be formulated into compositions for administration to subjects suffering from endocrine cancer. Therefore, the present invention also relates to a composition comprising kallikrein 13 or a fragment thereof, or an agent identified using a method of the invention, and a pharmaceutically acceptable carrier, excipient or diluent. A method for treating or preventing endocrine cancer in a subject is also provided comprising administering to a patient in need thereof hK5 or an agent identified in accordance with a method of the invention, or a composition of the invention.

The invention further provides a method of inhibiting endocrine cancer in a patient comprising:
(a) obtaining a sample comprising diseased cells from the patient;
(b) separately maintaining aliquots of the sample in the presence of a plurality of test agents;
(c) comparing-levels of kallikrein 13 in each aliquot;
(d) administering to the patient at least one of the test agents which alters the levels of the kallikrein 13 in the aliquot containing that test agent, relative to the other test agents.

In an embodiment, a test agent that decreases the levels of kallikrein 13 in an aliquot is administered to the patient. In another embodiment, a test agent that increases the levels of kallikrein 13 in an aliquot is administered to the patient.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies.

The therapeutic activity of antibodies specific for kallikrein 13, compositions, and compounds identified using a method of the invention and may be evaluated in vivo using a suitable animal model.

The following non-limiting examples are illustrative of the present invention:

### EXAMPLE 1

Materials and Methods

Cloning of KLK13 cDNA into the Yeast Expression System

Recombinant hK13 was produced using the Pichia pastoris yeast expression system (Invitrogen, Carlsbad, Calif., USA). Two primers were designed to amplify the KLK13 cDNA sequence: the forward primer was 5′ TCC AAG GAA TTC AAC ACC AAT GGG ACC 3′ [SEQ ID NO. 3] and the reverse primer was 5′CCA TTG TCT AGA TTG GGA CAT TCA GGT 3′ [SEQ ID NO. 4]. Human salivary cDNA was used as a template. PCR was carried out in a 20 μL reaction mixture, containing 1 μL of cDNA, 10 mM Tris-HCl (pH 8.3), 50 mM KCl, 1.5 mM MgCl₂, 200 μM deoxynucleoside triphosphates, 100 ng primers, and 2.5 units of Pfu DNA polymerase (Stratagene, La Jolla, Calif., USA) using an Eppendorf master cycler. The PCR conditions were 94° C. for 5 min, followed by 94° C. for 30 s, 62° C. for 30 s, 72° C. for 30 s and a final extension at 72° C. for 5 min. The PCR product was then cloned into the yeast expression vector pPICZαA using standard procedures (Sambrook J et al, 1989). The sequence of the construct was confirmed with an automated DNA sequencer.

Production of hK13 in Yeast

The pPICZαA vector containing the KLK13 cDNA sequence was introduced into the yeast strains X-33, KM71 and GS115. A stable clone was selected from the X-33 strain following the manufacturer's recommendations (Invitrogen). hK13 was produced by growing the stable yeast clone in a medium containing 10 g/L yeast extract, 20 g/L peptone, 100 mmol/L potassium phosphate (pH 6.0), 13.4 g/L yeast nitrogen base and 40 mg/L biotin in a 30° C. shaking incubator (250 rpm). hK13 production was induced with 5% methanol over 6 days. The cells were subsequently spun down, and the supernatant collected. An hK13 rabbit polyclonal peptide antibody (produced in-house) was used to monitor hK13 production by Western blot analysis.

Characterization of hK13 by Mass Spectrometry

Proteolytic digestion. Polyacrylamide gels were stained with Coomassie G-250 for visualization and selected bands were subsequently excised and destained with 300 mL/L acetonitrile in 100 mM ammonium bicarbonate. Each band was then reduced (10 mmol/L dithiothreitol in 50 mM ammonium bicarbonate, pH 8.3) and alkylated (50 mM iodoacetamide in 50 mmol/L ammonium bicarbonate, pH 8.3) before overnight trypsin digestion. Peptide fragments were then extracted with 50 mL/L acetic acid, evaporated dry on a Savant concentrator, and reconstituted in 10 μL of a solution of methanol-water-acetic acid (500:495:5 by volume).

All nanoelectrospray mass spectrometry experiments were conducted on a Q-star (PE/Sciex) hybrid quadrupole/time-of-flight instrument, for high resolution and online tandem mass spectrometry (MS-MS) experiments (Shevchenko, A et al, 1997). Conventional mass spectra were obtained by operating the quadrupole in a radiofrequency-only mode while a pusher electrode was pulsed at a frequency of approximately 7 kHz to transfer all ions to the time-of-flight analyzer. MS-MS experiments on trypsin-digested peptides identified in survey scans were conducted using a nanoelectrospray source. Precursor ions were selected by the first quadrupole while a pusher electrode was pulsed (frequency approximately 7 kHz) to transfer fragment ions formed in the radiofrequency-only quadrupole cell to the time-of-flight analyzer. Mass spectral resolution was typically 9000-10000. A scan duration of 1 and 2 s was set for conventional and MS-MS mass spectral acquisition, respectively. Collisional activation was performed using nitrogen collision gas with typically a 30-V offset between the DC voltage of the entrance quadrupole and the radiofrequency-only quadrupole cell. Data was acquired and processed using LC Tune and Biomultiview programs from PE/Sciex.

Purification of hK13 with Cation-Exchange and Reverse-Phase Chromatography

The recombinant hK13 was purified from yeast culture supernatants by cation-exchange chromatography using CM-Sepharose fast flow (Pharmacia Biotech, Piscataway, N.J.) and reverse-phase liquid chromatography using a C₄ column (0.45×5 cm; Vydac). The presence of hK13 in various fractions was identified with Western blotting using an anti-hK13 peptide antibody. In summary, the CM-Sepharose beads previously activated with 1M KCl were equilibrated in 10 mM MES buffer (pH 6.5). The yeast culture supernatant was first absorbed on CM-Sepharose beads by incubation at 4° C. overnight under agitation. The beads were then washed with 10 mM MES buffer (pH 6.5), and hK13 was eluted with 30 mM KCl in 10 mM MES buffer (pH 6.5). Trifluoroacetic acid as ion-pairing agent was added into this eluate (final concentration, 10 mL/L), which was then loaded on a C₄ column equilibrated with 1 mL/L trifluoroacetic acid in water. A linear gradient (1%/min) of acetonitrile from 15% to 50% in 1 mL/L trifluoroacetic acid was then performed. The fraction containing hK13 was evaporated on a SpeedVac (Savant). The purified material was then separated by sodium dodecyl sulfate-polyacrylamide gel electrophoresis (SDS-PAGE) and stained with Coomassie Blue to assess its purity and its molecular mass. The protein concentration of the purified hK13 was determined by the bicinchoninic acid method, which uses bovine serum albumin as calibrator (Pierce Chemical Co, Rockford, Ill.).

Assessment of hK13 Protein Glycosylation

hK13 protein glycosylation was assessed by treatment with the deglycosylation enzyme PNGaseF and the Gel Code glycoprotein staining kit following the manufacturer's recommendations (Pierce).

Assessment of hK13 Enzymatic Activity

The enzymatic activity of recombinant hK13 protein was assessed by using the fluorogenic substrate F—S—R-AMC (7-amido-4-methylcoumarin) and V—P—R-AMC. 10 mM Tris, pH 7.5, 0.1 NaCl, 0.5% bovine serum albumin was used as a buffer and 5 μL of 10 mM substrate solution, 1 μL of enzyme (1-600 ng) and 94 μL of buffer were mixed. The mixture was then incubated at 37° C. for 30 min and added 700 mL of 12.5% acetic acid to stop the reaction. The fluorescence was measured at an excitation wavelength of 300 nm and emission of 460 nm. Trypsin was used as the positive control. The fluorogenic substrates were purchased from Bachem, King of Prussia, PA, USA.

Amino-Terminal Sequencing of hK13 Recombinant Protein

Amino-terminal sequencing of purified recombinant hK13, blotted on polyvinyldifluoride membranes and stained with Coomassie blue, was performed with the Edman degradation method on an automated amino acid sequencer.

Production of Polyclonal Antibodies Against hK13

The purified recombinant hK13 protein was used as an immunogen to immunize rabbits and mice. HK13 (100 μg) was injected subcutaneously into Balb/C female mice and New Zealand white female rabbits. The protein was diluted 1:1 in complete Freund's adjuvant for the first injection and in incomplete Freund's adjuvant for the subsequent injections. Injections were repeated six times at 3-week intervals. Blood was drawn from the animals and tested for antibody generation. To test for production of anti-hK13 polyclonal antibodies in mice and rabbits (see below), the following immunoassay was used. Sheep anti-mouse or goat anti-rabbit IgG (Jackson ImmunoResearch, West Grove, Pa., USA) was immobilized on 96-well white ELISA plates. The mouse/rabbit serum was then applied to the plates at different dilutions ranging from 1:500 to 1:50000. After incubation (1 h) and washing, biotinylated recombinant hK13 was then added to each well (5-10 ng/well). Finally, after incubation (1 h) and washing, alkaline phosphatase-conjugated streptavidin was added, incubated (15 min), washed and the alkaline phosphatase activity was detected with time-resolved fluorescence (for details, see below).

Production and Characterization of Monoclonal Antibodies

Female Balb/c mice were immunized with recombinant hK13 protein. The immune splenocytes were fused with murine myeloma cells using standard hybridoma technology. Briefly, 100 μg (200 μL) of hK13 were diluted 1:1 in complete Freund's adjuvant for the first injection and in incomplete Freund's adjuvant for subsequent injections, and given subcutaneously, every 3 weeks, for three times. Two weeks after the third injection, the mouse was injected intraperitoneally with aqueous hK13, and three days later it was sacrificed and its spleen removed. To generate monoclonal anti-hK13 antibodies, the splenocytes were fused with the Sp2/0 myeloma cells using polyethylene glycol (PEG) 1500. The fused cells were cultured in 96-well plates in DMEM (Dulbecco's modified Eagle Medium) (Gibco BRL, Gaithersburg, Md.) containing 20% fetal calf serum, 200 mM glutamine, 1% OPI (oxaloacetic acid, pyruvic acid, insulin), and 2% HAT (hypoxanthine, aminopterin, thymidine; Sigma Chemical Co., St. Louis, Mo.) for selection at 37° C., 5% CO₂ for 10 to 14 days. The supernatants were collected and screened for positive clones using the following immunoassay. Sheep anti-mouse IgG, Fc fragment-specific antibody (Jackson Immmunresearch) was immobilized on 96-well white ELISA plates. Tissue culture supernatants diluted 10-fold in a general diluent (containing 60 g/L BSA, 50 mmol/L Tris, pH 7.80, and 0.5 g/L sodium azide) were applied to the plates, incubated for 1 h and washed 6 times. Biotinylated recombinant hK13 was then added (5-10 ng/well), incubated for 1 h and washed. Finally, alkaline phosphatase-conjugated streptavidin was added, incubated for 30 min and washed and the alkaline phosphatase activity was detected with time-resolved fluorescence, as described elsewhere (Christopoulos T K and Diamandis, 1992). The positive clones were expanded sequentially in 24 well plates and 6 well plates in complete media (reducing the fetal calf serum to 15% and changing the HAT to HT). Supernatants were further tested by performing IgG isotyping and clones were subjected to limiting dilution. The clones were then expanded in flasks to generate large amounts of supernatants in serum free-media (CD-1 media; Gibco BRL) containing 200 mM glutamine.

Purification of Monoclonal Anti-hK13 Antibodies

Tissue culture supernatants containing monoclonal antibodies were diluted 2-fold in 20 mM sodium phosphate buffer (pH 7.0) and injected into a HiTrap Protein G column (Pharmacia). After the column was washed with 20 mM sodium phosphate, the antibodies were eluted with 0.1 M glycine buffer (pH 2.7). The eluted antibody solutions were neutralized and then dialyzed overnight in 0.1 M sodium bicarbonate solution.

Immunofluorometric Assay for hK13

Standard assay procedure: For the polyclonal assay (mouse polyclonal/rabbit polyclonal), white polystyrene microtiter plates were coated with sheep anti-mouse IgG, Fc fragment-specific antibody (Jackson ImmunoResearch) by overnight incubation of 100 μL of coating antibody solution (containing 500 ng of antibody diluted in 50 mmol/L Tris buffer, pH 7.80) in each well. The plates were then washed six times with the washing buffer (9 g/L NaCl and 0.5 g/L Tween 20 in 110 mmol/L Tris buffer, pH 7.40). Mouse anti-hK13 polyclonal antiserum was diluted 500-fold in a general diluent [60 g/L bovine serum albumin, 50 mmol/L Tris (pH 7.80) and 0.5 g/L sodium azide], and 100 μL were applied to each well. After 1-h incubation, the plates were washed six times with washing buffer.

hK13 calibrators or samples were then pipetted into each well (50 μL/well along with 50 μL of the general diluent) and incubated for 1 h with shaking; the plates were then washed with washing buffer six times. Subsequently, 100 μL of rabbit anti-hK13 antiserum diluted 1000-fold in buffer A (containing the components of the general diluent plus 25 mL/L normal mouse serum, 100 mL/L normal goat serum, and 10 g/L bovine IgG) were applied to each well and incubated for 30 min; plates were then washed as described earlier. Finally, 100 μL/well of alkaline phosphatase-conjugated goat anti-rabbit IgG, Fc fragment-specific (Jackson ImmunoResearch), diluted 2000-fold in buffer A were added to each well and incubated for 36 min, and plates were washed as above.

Diflunisal phosphate (100 μL of a 1 mmol/L solution) in substrate buffer (0.1 mol/L Tris, pH 9.1, 0.1 mol/L NaCl, and 1 mmol/L MgCl₂) was added to each well and incubated for 10 min. Developing solution (100 μL, containing 1 mol/L Tris base, 0.4 mol/L NaOH, 2 mmol/L TbCl₃, and 3 mmol/L EDTA) was pipetted into each well and mixed for 1 min. The fluorescence was measured with a time-resolved fluorometer, the Cyberfluor 615 Immunoanalyzer (MDS Nordion, Kanata, ON, Canada). The calibration and data reduction were performed automatically, as described elsewhere (Christopoulos T K and Diamandis E P, 1992). For the monoclonal antibody assay (two monoclonal antibodies), white polysterene microtiter plates were coated directly with one monoclonal antibody specific for hK13 (code 2-17; 500 ng/well). The plates were then washed six times with the washing buffer. Subsequently, hK13 calibrators were added along with a second biotinylated monoclonal antibody (code 11C1; 50 ng/well). Detection was achieved by using alkaline phosphatase-conjugated streptavidin. Diflusinal phosphate and developing solution were added as stated above. For the monoclonal/polyclonal immunoassay configuration, mouse monoclonal ¹¹C1 was used for directly coating microtiter plates and the rabbit polyclonal antibody was used for detection, with the procedures described above. More details about such procedures were described elsewhere for similar immunoassays for hK6 (Diamandis E P et al, 2000) and hK10 (Luo L Y et al, 2001).

Determination of the sensitivity of the hK13 immunoassay: Recombinant hK13 was used to generate the calibration curve. hK13 calibrators were prepared by diluting the purified recombinant hK13 in the general diluent. These calibrators were then used to define the detection limit of the assay.

Determination of the specificity of the hK13 immunoassay: Biological fluids and recombinant hK13 were used to determine the specificity of the developed immunoassay. These samples were first measured by the standard assay procedure described above. The mouse and rabbit anti-hK13 antisera were then successively replaced with sera from the same animals, obtained before immunization (preimmune sera). The samples were measured again, and the fluorescence counts were compared with the counts obtained by the standard assay. The cross-reactivities of other homologous proteins were investigated using purified recombinant hK1-hK15, all diluted in the general diluent.

Human Tissue Cytosolic Extracts and Biological Fluids

Human tissue cytosolic extracts were prepared as follows: Various frozen human tissues (0.2 g) were pulverized on dry ice to fine powders. Extraction buffer (1 mL, containing 50 mmol/L Tris, pH 8.0, 150 mmol/L NaCl, 5 mmol/L EDTA, 10 g/L NP40 surfactant, 1 mmol/L phenylmethylsulfonyl fluoride, 1 g/L aprotinin, 1 g/L leupeptin) was added to the tissue powders, and the mixture was incubated on ice for 30 min with repeated shaking and vortex-mixing every 10 min. Mixtures were then centrifuged at 14,000 g at 4° C. for 30 minutes. The supernatants (cytosolic extracts) were then collected. The biological fluids were leftovers of samples submitted for routine biochemical testing. All tissue cytosolic extracts and biological fluids were stored at −80° C. until use.

Recovery

Recombinant hK13 was added to the general diluent (control), to male and female normal sera and various biological fluids at different concentrations and the spiked samples were measured with the monoclonal-monoclonal, monoclonal-polyclonal and polyclonal-polyclonal hK13 immunoassay. Recoveries were then calculated after subtraction of the endogenous concentrations.

Fractionation of Biological Fluids with Size-Exclusion HPLC

To determine the molecular mass of the protein detected in the biological fluids and tissue extracts, various samples were fractionated with gel filtration chromatography, as described elsewhere (Diamandis E P et al, 2000; Luo L Y et al, 2001). The fractions were collected and analyzed for hK13 using the developed immunoassay.

Immunohistochemistry

Rabbit polyclonal antibody was raised against full-length hK13, as described above. Immunohistochemical staining for hK13 was performed according to a standard immunoperoxidase method. Briefly, paraffin-embedded tissue sections (4 μm) were fixed and dewaxed. Endogenous peroxidase activity was blocked with 3% aqueous hydrogen peroxide for 15 min. Sections were then treated with 0.4% pepsin at pH 2.0 for 5 min at 42° C. and blocked with 20% protein blocker (Signet Labs) for 10 min. The primary antibody was then added at 1:1000 dilution for 1 h at room temperature. After washing, biotinylated anti-rabbit antibody (Signet Labs) was added, diluted 4-fold in antibody dilution buffer (Dako). Following incubation and washing, streptavidin-tagged horseradish peroxidase was added for 30 min at room temperature. After washing, detection was achieved with diaminobenzidine (DAB) for 5-10 min. The slides were then counterstained with hematoxylin and then mounted with cover slips.

Results

Production, Purification and Characterization of Recombinant hK13 Protein

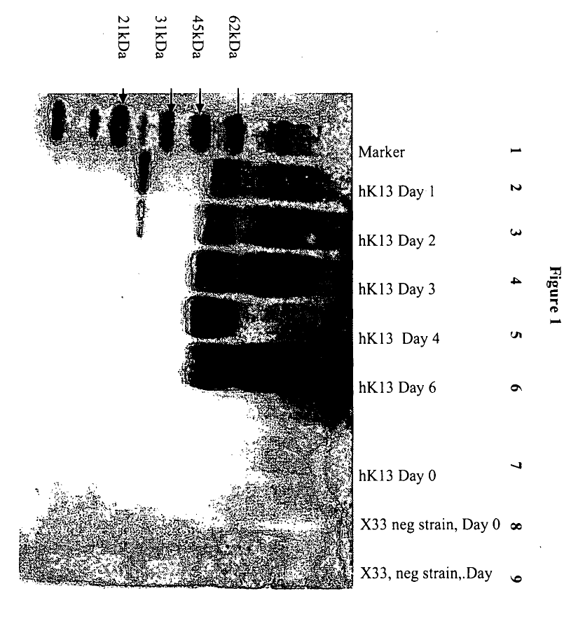

hK13 is predicted to be a secreted serine protease. Hydrophobicity and structural homology analysis suggested that the active form of hK13 starts from amino acid 26 (the segment 1-20 represents the signal peptide and segment 21-25 is the activation peptide) (Yousef G M et al, 2000). The cDNA encoding for this active form of hK13 was cloned into a P. pastoris yeast expression system. Expression in yeast produced a higher molecular mass protein (˜50 kDa) and much smaller amounts of a 28 kDa protein. Both proteins were visible on Western blots, as shown in FIG. 1. The bands representing the 50 kDa and 28 kDa proteins were excised from Coomassie-stained gels and subjected to mass spectrometric analysis. Selected trypsin digested fragments were then sequenced by using tandem mass spectrometry. These data demonstrated that both bands represent hK13 protein. For example, the partial sequence VSGWGTTTSPQVNYPK [SEQ ID NO. 5] was identified from the peptide fragments and precisely matched the fragment expected for amino acids 159-174 of hK13 (Yousef G M et al, 2000).

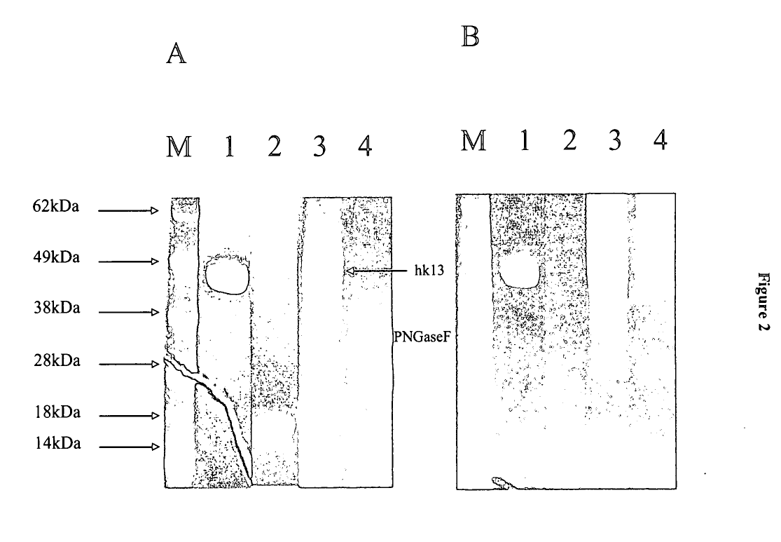

The higher molecular weight recombinant hK13 protein is probably highly glycosylated. The protein was separated on SDS-PAGE gels and stained with Coomassie blue before and after digestion with the diglycosylating enzyme PGNaseF (FIG. 2A). Upon treatment with PGNaseF, the molecular weight of the recombinant hK13 protein is reduced to approximately 25-28 kDa, consistent with the molecular weight of the non-glycosylated hK13 protein (Yousef G M et al, 2000). To further verify these findings, recombinant hK13 was stained with acidic fuchsin sulfite (this stain is specific for glycoproteins only) before and after treatment with PGNaseF. It is shown that after treatment with PNGaseF, the staining of the glycosylated hK13 is abolished (FIG. 2B).

In order to verify the starting amino acid of the purified recombinant hK13 protein, hK13 was purified by using cation-exchange chromatography and reverse-phase chromatography, as described in ‘Methods’. The amino terminal end was sequenced by the Edman degradation method. The amino terminal end of this protein is represented by the amino acids VLNTNG [SEQ ID NO. 6]. These data confirm that the protein represents the active form of the enzyme since activation occurs after cleavage of a K ⇓V bond between amino acids 25 and 26 of the pre-proenzyme (Yousef G M and Diamandis E P, 2000, Yousef G M et al, 2000).

Recombinant hK13 protein was further verified to have enzymatic activity. Two trypsin substrates, FSR-AMC and VPR-AMC were used. Trypsin was highly active with both fluorogenic substrates, even at amounts as low as 1 ng. hK13 was also able to cleave both substrates in a dose-dependent manner. For example, under the experimental conditions outlined, hK13 activity resulted in an increase of fluorescence of the substrate FSR-AMC from 1.1 arbitrary units to 1.9 arbitrary units (10 ng of hK13), to 6.9 arbitrary units (100 ng of hK13) and to 14.5 arbitrary units (160 ng of hK13). For the substrate VPR-AMC, the enzyme was able to increase the fluorescence of the substrate from 0.5 arbitrary units to 1.9 arbitrary units (10 ng of enzyme) to 13.9 arbitrary units (100 ng of enzyme) and to 35.5 arbitrary units (160 ng of enzyme).

Production of Antibodies and Development of the Immunofluorometric Assay

Recombinant hK13 protein was used as an immunogen in mice and rabbits to generate polyclonal and monoclonal antibodies. These antibodies were then used for immunohistochemical localization of hK13 (see below) and for the development of three different versions of an ELISA-type immunofluorometric assay. Among the three assay configurations (polyclonal mouse coating antibody/polyclonal rabbit detection antibody; monoclonal mouse coating antibody/polyclonal rabbit detection antibody; monoclonal mouse coating antibody/biotinylated monoclonal mouse detection antibody), the most sensitive was based on a monoclonal/polyclonal configuration. In selected cases, all three research configurations were used. Below, is described the characteristics of the developed ELISA-type immunofluorometric procedure.

Sensitivity, Specificity, Linearity and Precision of the hK13 Immunofluorometric Assay

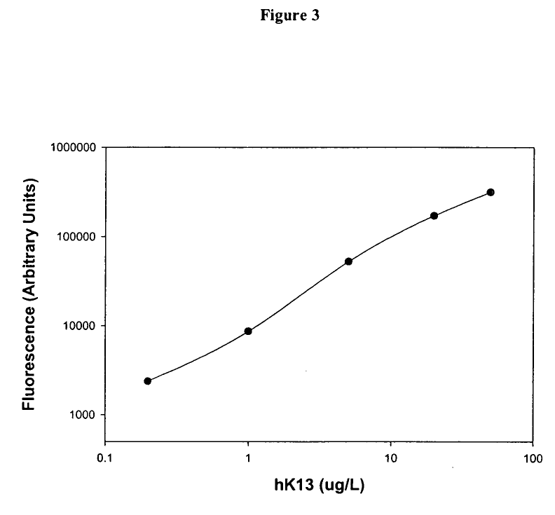

Sesitivity: A typical calibration curve for the hK13 immunofluorometric assay is shown in FIG. 3. The detection limit, defined as the concentration of hK13 that can be distinguished from zero with 95% confidence, was 0.05 μg/L. These data were obtained with the mouse monoclonal (11CI)/rabbit polyclonal immunoassay configuration, which was more sensitive than the other two assay configurations (monoclonal/monoclonal and polyclonal/polyclonal immunoassays).

Specificity: For all assay configurations, it was confirmed that the assay specifically measures hK13. When mouse and rabbit antisera were replaced with pre-immune mouse and rabbit sera, the fluorescence signals of hK13-positive samples were reduced to nearly zero (data not shown). Because hK13 is a member of the human kallikrein family, it shares significant amino acid homology with other kallikreins. To demonstrate that there is no interference from these homologous proteins, the cross-reactivities of recombinant hK1, hK2, hK3, hK4, hK5, hK6, hK7, hK8, hK9, hK10, hK11, hK12 hK14 and hK15 were examined. None of these recombinant proteins produced measurable readings, even at concentrations 1000-fold higher than hK13. These data suggest that this immunoassay can efficiently discriminate hK13 from other homologous proteins and that it measures hK13 with high specificity.

Linearity and precision: To assess the linearity of this assay, various samples were diluted serially and hK13 was re-measured. Samples included milks, seminal plasmas and amniotic fluids. A good dilution linearity was found with this assay, suggesting freedom from matrix effects. Within- and between-run precision was assessed with various hK13 calibrators and clinical samples. In all cases, the CVs were between 3-8%.

Distribution of hK13 in Various Human Tissue Extracts

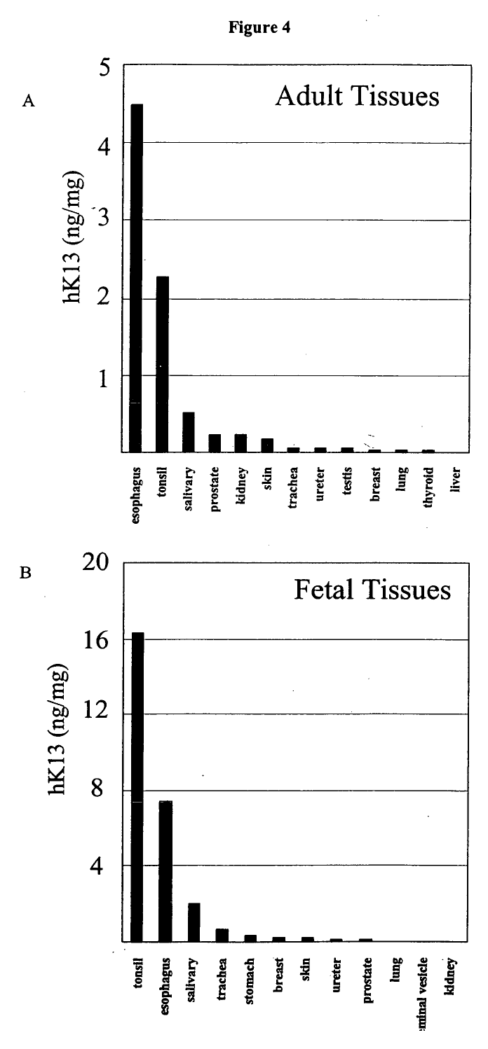

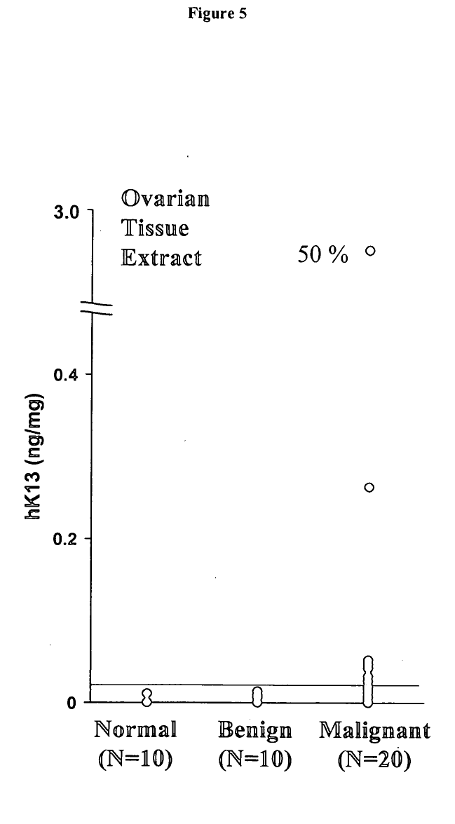

The distribution of hK13 in various human tissue extracts (adult and fetal) was investigated with the developed hK13 immunoassay. The data are graphically shown in FIG. 4. Highest levels of hK13 were seen in esophagus and tonsil, followed by salivary glands, prostate, kidney, skin, trachea, ureter, testis, breast, lung and thyroid. A similar pattern was also seen in fetal tissues (FIG. 4).

hK13 in Biological Fluids

hK13 protein was quantified in various biological fluids, as shown in Table 1. Highest levels were seen in seminal plasma, followed by amniotic fluid, breast milk and follicular fluid. Cerebrospinal fluid contains only traces of hK13 and all male and female serum samples from normal individuals were negative for hK13 (concentration below 0.05 μg/L).

Ovarian Cancer Cytosols and Ascites Fluids

Since multiple kallikreins have already been shown to be overexpressed in ovarian carcinoma, the concentration of hK13 was examined in ovarian cancer cytosolic extracts. Among 20 extracts, 10 (50%) were positive for hK13, with concentrations ranging from 0.2 to 15 μg/L. None of the 10 extracts from either normal ovarian tissues or benign ovarian disease were highly positive (FIG. 5). The data of FIG. 5 have been normalized for total protein content. 44 ascites fluid samples from women with advanced ovarian carcinoma were also tested. All ascites samples were positive for hK13, with values ranging from 0.1 to 20 μg/L. The mean±standard deviation and median values were 2.3±4.2 and 0.8 μg/L, respectively.

Recovery of hK13 from Biological Fluids

The recovery of added recombinant hK13 in amniotic fluid and milk samples was tested. Recoveries ranged from 70 to 98%. When the same experiments were performed with male and female sera, recovery ranged from 5 to 10%. This low recovery was verified with all three versions of the developed immunoassay. About the same recovery (5-10%) was obtained when serum samples from males and females were spiked with hK13 from amniotic fluid and seminal plasma (data not shown). These data prompted us to speculate that hK13 may be quickly sequestered by proteinase inhibitors present in serum, similarly to other kallikreins, including hK3 (PSA) and hK2 (Stenman U H et al, 1991; Lilja H et al, 1991; Stephan C. et al, 2000; Saedi M S et al, 2001).

Fractionation of Biological Fluids with Size-Exclusion HPLC

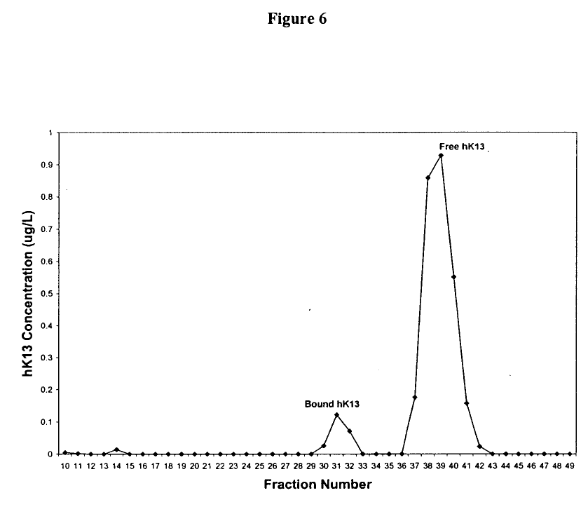

To determine the molecular mass of the protein detected in tissue extracts and the biological fluids, selected samples were fractionated on a gel filtration column. The presence of hK13 in various fractions was then assessed with the developed immunoassay. When the hK13 concentration in fractions were plotted against the fraction number, a peak around fraction 38 (corresponding to a molecular mass of approximately ˜30 kDa) was consistently detected. This single peak was seen with an esophageal extract, seminal plasma, amniotic fluid and follicular fluid. These results indicate that the protein detected with the hK13 immunoassay is a single species, with a molecular mass of ˜30 kDa, which is consistent with the molecular mass of free (non-complexed) hK13. However, in ascites fluid from ovarian cancer patients, a small peak corresponding to a molecular mass of −100 kDa was detected. This peak likely represents hK13 bound to a proteinase inhibitor (FIG. 6).

Immunohistochemistry

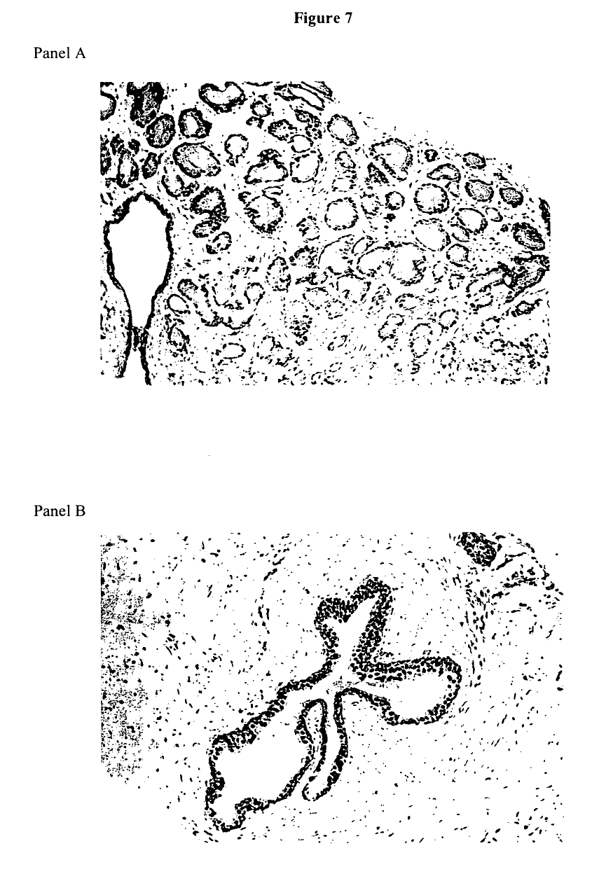

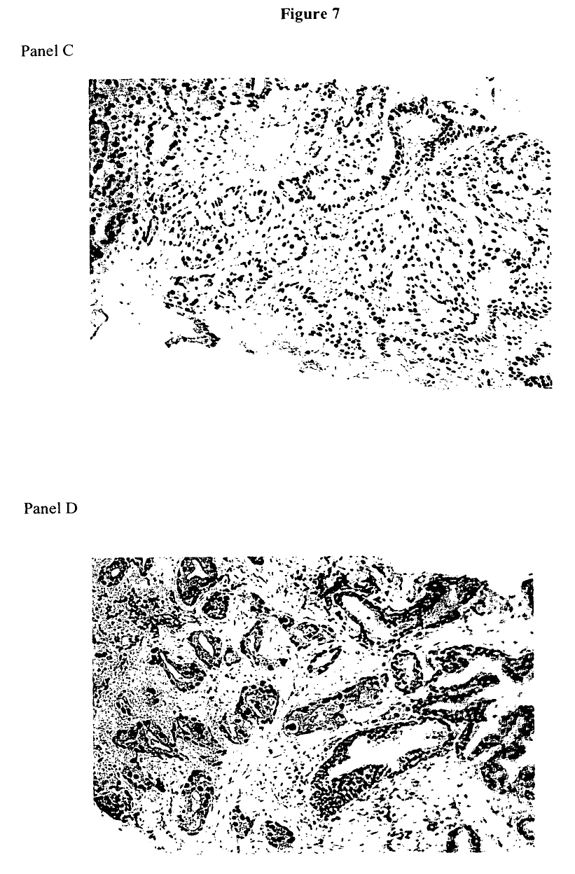

By using standard immunohistochemical procedures, hK13 was localized in both non-malignant and malignant prostatic tissues (FIG. 7). Staining was mostly cytoplasmic in epithelial cells, while stroma was negative. Some nuclear staining of epithelial cells was also evident.

Discussion

The KLK13 gene was cloned using the positional candidate approach and found to be down-regulated in breast cancer tissues and breast cancer cell lines (Yousef G M et al, 2000). On the basis of its chromosomal location and structural similarities with other kallikreins, KLK13 was classified as a novel member of the kallikrein gene family (Diamandis E P et al 2000; Yousef G M et al, 2001; Yousef G M et al, 2000). This study has developed reagents and methods to detect hK13 in tissues and fluids. Because there is no known natural source of hK13, it was expressed in relatively large amounts in a Pichia pastoris expression system. It was then purified to homogeneity using ion-exchange and reverse-phase chromatography. The recombinant hK13 protein was positively identified by mass spectrometry. Compared to the predicted molecular mass of hK13 (Yousef G M et al, 2000), the protein in yeast is found in two forms, non-glycosylated (˜28 kDa) and heavily glycosylated (50 kDa), the latter being the predominant species. This protein represents the active form of the enzyme and has trypsin-like cleavage specificity, as demonstrated by synthetic substrates.

Initially, mouse and rabbit polyclonal antibodies against recombinant hK13 were produced and used to develop a first-generation immunological assay for hK13. By using this assay, it was confirmed that hK13 is a secreted protein, found in various biological fluids. Subsequently, monoclonal antibodies against hK13 were generated and two hybridoma cell lines producing antibodies that could be incorporated into a “sandwich-type” assay were isolated. The most sensitive assay was based on a mouse monoclonal coating/rabbit polyclonal detection antibody configuration. This assay is highly sensitive and specific, detecting hK13 at concentrations of 0.05 μg/L or higher. No detectable cross-reactivity to other recombinant human kallikreins (hK2 to hK15) was found. Size-exclusion HPLC of biological fluids further indicated that this assay detects only one immunoreactive peak of the expected molecular mass (˜30 kDa). These data suggest that the immunoassay detects the free fraction of hK13, although in ascites fluid from an ovarian cancer patient, there is another peak, likely representing hK13, bound to a proteinase inhibitor or another interacting protein.

hK13 was detected in various tissue extracts but predominantly in esophageal, tonsil, salivary; prostate, kidney and skin extracts. hK13 was also identified in seminal plasma, amniotic fluid, follicular fluid and breast milk, suggesting that this enzyme is secreted by cells of male and female reproductive organs. The enzyme was immunohistochemically localized in prostatic tissues (FIG. 7). Unlike other kallikreins (e.g. hK6, hK10) (Diamandis B P et al, 2000; Luo L Y et al, 2001), hK13 is not present at appreciable amounts in CSF. Similarly, the concentration of hK13 in serum of healthy men and women appears to be very low, below the detection limit of the assay (<0.05 μg/L).

The immunoassay revealed that the recovery of recombinant hK13 from serum was incomplete with values ranging from 5-10%. Differences between the monoclonal-monoclonal, monoclonal-polyclonal and polyclonal-polyclonal immunoassay configurations were not observed. Serum may contain proteinase inhibitors or other interacting proteins that bind to free hK13. The recombinant protein is enzymatically active and may be sequestered by excess proteinase inhibitors such as CL₂-macroglobulin. It is already well known that another two kallikreins, hK3 (PSA) and hK2 form complexes with many proteinase inhibitors in serum (Stephan C et al, 2000; Saedi M S et al, 2001; Zhang W M et al, 1998; Zhou A M et al, 1993; Grauer L S et al, 1998; Grauer, L S et al, 1996; Deperthes D et al, 1995; Mikolajczyk S D et al, 1999).

Herein is presented the first evidence that hK13 is overexpressed in a subset of patients with ovarian carcinoma (FIG. 5). Relatively high levels of hK13 were found in ascites fluid of women with ovarian cancer. Thus, hK13 is a kallikrein that is overexpressed in ovarian carcinoma.

### EXAMPLE 2

Materials and Methods

Ovarian Cancer Patients and Specimens

One hundred and thirty-one patients with primary epithelial ovarian cancer were examined in this study, ranging in age from 20 to 85 and years, with a median age of 57 (Table 2). Patients were monitored for survival and disease progression (no apparent progression or progression) for a median duration of 42 months. Follow-up information was available for 131 patients, among which 74 (56%) had relapsed and 54 (41%) had died.

Histological examination, performed during intra-surgery frozen section analysis, allowed representative portions of each tumor containing more than 80% tumor cells to be selected for storage until analysis. Clinical and pathological information documented at the time of surgery included tumor stage, grade, histotype, residual tumor size, debulking success and volume of ascites fluid (Table 3). The staging of tumors was in accordance with the FIGO criteria (Pettersson F, 1994), grading was established according to Day et al. (Day T G et al, 1975) and the classification of histotypes was based on both the WHO and FIGO recommendations (Serov, S F et al, 1973).

Patients with disease at clinical stages I-IV and grades (1-3) were represented in this study. Of the 134 ovarian tumors, the majority (93; 71%) were of the serous papillary histotype, followed by mucinous (12; 9%), undifferentiated (11; 8%), endometrioid (6; 5%), clear cell (4; 3%) or were unclassified (5; 4%). The residual size of tumors ranged from 0 to 6 cm.

Investigations were carried out in accordance with the ethical standards of the Helsinki Declaration of 1975, as revised in 1983 and were approved by the Institutional Review Boards of Mount Sinai Hospital and the Technical University of Munich.

Preparation of Cytosolic Extracts

Tumor specimens were snap-frozen in liquid nitrogen immediately after surgery and stored at −80° C. until extraction. Frozen tissues (20-100 mg) were pulverized on dry ice to a fine powder and added to 10 volumes of extraction buffer (50 mM Tris, pH 8.0, 150 mM NaCl, 5 mM EDTA, 10 g/L of NP-40 surfactant, 1 mM phenylmethylsulfonyl fluoride, 1 g/L of aprotinin, 1 g/L of leupeptin). The resulting suspensions were incubated on ice for 30 minutes, with repeated shaking and vortexing every 10 minutes. The mixtures were then centrifuged at 14,000 rpm at 4° C. for 30 minutes and the supernatant (cytosolic extract) was collected and stored at −80° C. until further analysis. Protein concentration of the extracts was determined using the bicinchoninic acid method, with bovine serum albumin (BSA) as standard (Pierce Chemical Co., Rockford, Ill.).

Measurement of hK13 in Ovarian Cytosolic Extracts

The concentration of hK13 in cytosolic extracts was quantified using a highly sensitive and specific non-competitive “sandwich-type” immunoassay, previously described and evaluated (See Example 1). Briefly, microtiter plates were coated directly with a mouse anti-hK13 monoclonal antibody (code 2-17; 500 ng/well). After a 1 h incubation, the plates were washed 6 times with washing buffer (9 g/L NaCl and 0.5 g/L Tween 20 in 10 mmol/L Tris buffer, pH 7.40). Then, either recombinant hK13 calibrators [50 μL/well and 50 μL of a general diluent (60 g/L BSA, 50 mmol/L Tris, pH 7.80, 0.5 g/L sodium azide)] or cytosolic extracts (50 μL/well and 50 μL of the general diluent) were applied to each well in duplicate, incubated for 2 hours with gentle shaking and washed. Rabbit anti-hK13 polyclonal antiserum (diluted 1000-fold in assay buffer containing the components of the general diluent plus 25 mL/L normal mouse serum, 100 mL/L normal goat serum and 10 g/L bovine IgG) was subsequently applied, incubated for 1 hour and washed. Finally, alkaline phosphatase-conjugated goat anti-rabbit IgG (Jackson Immunoresearch, West Grove, Pa.), diluted 2000-fold in assay buffer, was added, incubated for 45 min and washed as before. Signal detection and data reduction were performed automatically by the CyberFluor 615 Immunoanalyzer, which uses time resolved fluorometry, as described elsewhere (Christopoulos T K and Diamandis E P, 1992). The detection range of this assay is 0.1-20 μg/L. hK13 measurements in μg/L were converted to ng of hK13/mg of total protein to adjust for the amount of tumor tissue extracted.

Statistical Analysis

Statistical analyses were performed with SPSS software (SPSS Inc. Richmond, Calif.). The hK13 status of ovarian tumour extracts was categorized as either hK13-positive or hK13-negative. The relationship between hK13 status and various clinicopathological variables was analyzed with the χ² test and the Fisher's exact test, as appropriate.

For survival analysis, two different end points—cancer relapse (either local recurrence or distant metastasis) and death—were used to calculate progression free (PFS) and overall survival (OS), respectively. PFS was defined as the time interval between the date of surgery and the date of identification of recurrent of metastatic disease. OS was defined as the time interval between the date of surgery and the date of death. The impact of hK13 on patient survival (PFS and OS) was assessed with the hazard ratio (relative risk of relapse or death in the hK13-positive group) calculated with the Cox univariate and multivariate proportional hazard regression model (Cox D R, 1972). Only patients for whom the status of all variables was known were included in the multivariate regression models. The multivariate models were adjusted for hK13 expression in tumors and other clinical and pathological variables that may affect survival, including age, stage of disease, tumor grade, CA125 and age. Kaplan-Meier PFS and OS curves (Kaplan E L and Meier P, 1958) were also constructed in order to demonstrate survival differences between the hK13-positive and hK13-negative patients. The differences between the survival curves were tested for statistical significance using the log rank test (Mantel, N, 1966).

Results

Distribution of hK13 Concentration in Ovarian Tumor Tissues

hK13 concentration in ovarian tumor cytosols from 131 patients ranged from 0 to 18.4 ng/mg of total protein, with a mean of 0.61 ng/mg total protein and a median of 0 ng/mg total protein (Table 2). An optimal cutoff value of 0.13 ng/mg total protein was identified by χ² analysis, based on the ability of hK13 to predict the PFS of the study population. Based on this cut-off (67^{th} percentile), 33% of the ovarian tumors were categorized as hK13-positive.

Relationships Between hK13 Status and Other Clinincopathiological Variables

The distributions of various clinicopathological variables between bK13-postitive and hK13-negative patients are summarized in Table 3. The relationships between hK13 and these variables were examined with either the χ² or Fisher's Exact Test. Patients with hK13-positive ovarian tumors were more likely to have early stage (Stage I/II) disease, no residual tumor and optimal debulking success (p<0.05). Although marginally significant, hK13-positive tumors were mainly of the clear cell and mucinous histotypes (p=0.089). No relationship was observed between hK13 status and tumor grade or volume of ascites fluid.

Univariate and Multivariate Survival Analysis

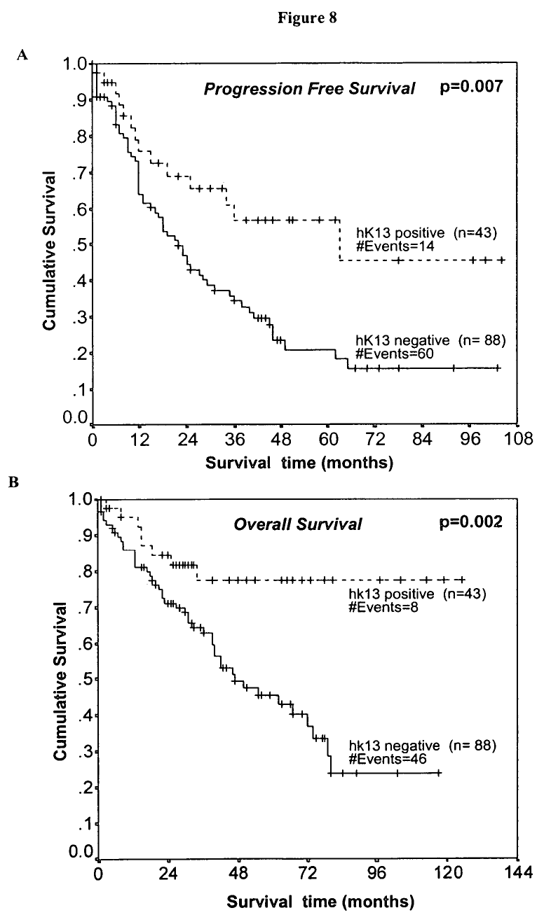

The strength of association between hK13-positive tumors and survival outcome is presented in Table 4. In univariate Cox regression analysis, hK13-positive patients had a lower risk of relapse (HR of 0.46, p=0.009) and death (HR of 0.33, p=0.004). Similarly, in multivariate Cox regression analysis, hK13 positivity was found to be significantly associated with a longer PFS and OS(HR of 0.53 and 0.34; p=0.042 and p=0.01, respectively). This regression model suggests that there is approximately a 47-66% reduction in either the risk of relapse or death in patients with hK13-positive tumors, compared to those who are hK13-negative. Kaplan-Meier survival curves (FIG. 8) further demonstrate that women with hK13-positive ovarian tumors have substantially longer PFS and OS (p<0.01), compared to those with hK13-negative tumors. As expected, disease staging was found to be strongly associated with decreased PFS and OS, in both univariate and multivariate analyses (p<0.001).

Discussion

The last few years, a plethora of studies have been published which attempt to refine our understanding of determinants of prognosis in ovarian cancer by analysing tumor-associated markers thought to be of biological relevance in the carcinogenic process. Proteases, of several catalytic types (serine, cysteine, metallo), are among these prognostic factors (Duffy M J, 1996). In the present study, the expression of a serine protease, hK13, has been evaluated in epithelial ovarian tumors in relation to other established prognostic indicators and patient survival. hK13-positive ovarian tumors were most frequently found in patients with early stage disease, no residual tumor and optimal debulking success. hK13 is an independent predictor of favourable prognosis in ovarian cancer, as evidenced by multivariate Cox proportional hazards regression analysis and Kaplan-Meier survival curves.

Typically, the expression of proteases in cancer tissues correlates with poor patient prognosis in different malignancies (Duffy M J, 1999; Vihinen, P, 2002; Kos J and Lah T T, 1998; Harbeck, N et al, 2001; Seetoo D Q et al, 2003; Foekens J A et al, 2003), including ovarian cancer (Ghosh, S et al, 2002; Konecny G et al, 2001; Lengyel, E et al, 2001; Wu X et al, 2002; Sui L et al, 2002) likely due to the well established roles of proteolytic enzymes in extracellular matrix (ECM) degradation, which facilitates invasion and metastasis (Duffy M J, 1991; Del Rosso et al, 2002). However, in recent years, with the identification of non-ECM substrates for secreted proteases, new roles for proteolytic enzymes have emerged in the regulation of cellular functions during tumor development, including cell proliferation, differentiation, survival, genomic (in)stability and angiogenesis (Noel, A et al, 1997; DeClerck Y A et al, 1997; Aimes R T et al, 2003). In fact, it has been documented that certain proteases, including matrix metalloproteinase-19 (Impola, U et al, 2003), and serine proteases such as, testisin (Hooper J D et al, 1999), prostasin (Chen L M and Chai K X, 2002; Takahashi S et al, 2003) and human kallikreins 3, 4, 5, 10, 12, 13 and 14 (Yousef G M and Diamandis E P, 2001), are down-regulated in hormone-dependent cancers, and several may function as tumor suppressors (Chen L M et al., 2001; Balbay M D et al, 1999; Goyal, J et al, 1998). These findings may help to explain why certain proteases, including hK13, are associated with a favourable prognosis in cancer patients (Scorilas A et al, 1999; Scorilas A, et al, 2001; Rojo J V et al, 2002; Hsia J Y et al, 2003).

Accumulating evidence suggests that at least 10 of the 15 human kallikrein family members have prognostic value in ovarian cancer (listed in Table 5), exclusive of hK13 (Yousef G M and Diamandis E P, 2002). Among these, kallikreins 8, 9, 11 and 14, are comparable to hK13 since they are most often expressed in early stage ovarian tumors and correlate with a favourable patient prognosis (Magklara A et al, 2001; Yousef G M et al, 2001; Borgono C A et al, 2003; Yousef G M et al, 2003). The remainder, kallikreins 4, 5, 6, 7, 10 and 15, are expressed in advanced ovarian tumors and are markers of poor prognosis (Obiezu C V et al, 2001; Dong Y et al, 2001; Kim H et at, 2001; Hoffman B R et al, 2002; Kyriakopoulou L G et al, 2003; Luo L Y et al, 2001; Yousef G M et al, 2003). Since the vast majority of kallikreins are co-expressed, and likely coordinately regulated in ovarian cancer, they may represent an enzymatic pathway involved in ovarian carcinogenesis (Yousef G M et al, 2002).

In contrast to earlier studies, which reported high kallikrein 4, 6, and 10 expression in serous epithelial ovarian tumors (Dong Y et al, 2001; Hoffman B R et al, 2002; Luo L Y et al., 2001), hK13-positive tumors were more frequently of the non-serous (i.e. clear cell and mucinous) histotypes (Table 5). Similar findings were obtained with kallikreins 5 and 11, which are also associated with non-serous tumors (i.e. undifferentiated and mucinous, respectively) (Borgono C A et at, 2003). This data suggests that hK13, together with hK5 and hK11, may be clinically useful as determinants of prognosis in the subgroup of ovarian cancer patients with non-serous epithelial tumors.

KLK13, at the mRNA level, is an independent marker of favourable prognosis in women with breast carcinomas (Chang A et al, 2002). The underlying biological mechanism of hK13 involvement in the progression of breast and ovarian cancers may be related to steroid hormones. First, hK13 is encoded by an androgen-regulated gene (Yousef G M et at, 2000). Second, epidemiological and experimental evidence suggests that steroid hormones, such as androgens, are implicated in the etiology of both breast and ovarian carcinomas (Henderson B E and Feigelson H S, 2000; Liao D J et al, 2002; Risch H A, 1998). Third, it has been documented that the androgen receptor (AR) is present in over 80% of breast (Isola J J, 1993) and in 84% of ovarian tumors (Cardillo, M R et al 1998) and that androgens, acting through the AR, can stimulate and inhibit breast (Liao D J and Dickson R B, 2002) and increase ovarian (Risch H A, 1998) cancer cell proliferation. Androgen-AR complexes may regulate KLK13 gene expression during breast and ovarian carcinogenesis. hK13, under androgenic stimulation, may inhibit breast and ovarian cancer metastasis in early stage carcinomas, by initiating or terminating events through the activation of favourable proteins (i.e. inhibitors) or cleavage of unfavourable ones (i.e. growth factors). Identification of downstream AR-regulated genes, such as KLK13, is also important in understanding the mechanism by which androgens are implicated in hormone-related malignancies.

In conclusion, this is the first report to describe the clinical utility of hK13 as an independent indicator of favourable prognosis in ovarian cancer patients. By virtue of its predominance in non-serous ovarian tumors, hK13 may also be particularly applied clinically in the corresponding subgroup of patients.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a cell” includes a plurality of such cells, reference to “peptide” is a reference to one or more peptide molecules and equivalents thereof known to those skilled in the art, and so forth.

Below full citations are set out for the references referred to in the specification.

TABLE 1
Analysis of hK13 in various biological fluids

| | | | | | Positivity |
|---|---|---|---|---|---|
| Biological Fluid | N¹ | Range (ug/L) | Mean (SD) | Median | rate |
| Seminal plasma | 10 | 86-406 | 274 (108) | 284 | 100% |
| Amniotic Fluid | 20 | 16-88 | 45 (20) | 41 | 100% |
| Breast milk | 10 | 3.8-30 | 8.6 (7.9) | 5.9 | 100% |
| Follicular fluid | 6 | 0.08-5.6 | 1.5 (1.0) | 0.81 | 100% |
| CSF | 10 | 0-0.24 | 0.15 (0.14) | 0.09 | 90% |
| Male/female sera | | 0 | 0 | 0 | 0% |
| (21/21) | | | | | |

¹Number of samples
2. CSF, cerebrospinal fluid

TABLE 2
Descriptive statistics of the continuous variables in the ovarian cancer study population

| | | | Percentiles | | | | | | |
|---|---|---|---|---|---|---|---|---|---|
| | | | | | | 50 | | | |
| Variable | Mean ± SE^{a} | Range | 10 | 25 | 40 | (Median) | 60 | 75 | 90 |
| hK13 (ng/mg) | 0.61 ± 0.19 | 0.00-18.4 | 0.00 | 0.00 | 0.00 | 0.00 | 0.0043 | 0.25 | 1.2 |
| CA 125 (KU/mg) | 2.7 ± 0.4 | 0.00-32.7 | 0.02 | 0.14 | 0.46 | 1.05 | 1.55 | 2.94 | 7.2 |
| Age (years) | 58 ± 1.1 | 20-85 | 41 | 51 | 55 | 57 | 62 | 68 | 76 |

^{a}Standard error

TABLE 3
Relationship between hK13 status¹ and other
variables in 131 ovarian cancer patients

| | | No. of patients (%) | | |
|---|---|---|---|---|
| Variable | Patients | hk13-negative | hK13-positive | p value |
| Stage | | | | |
| I/II | 32 | 15 (46.9) | 17 (53.1) | 0.009^{a} |
| III/IV | 99 | 73 (73.7) | 26 (26.3) | |
| Grade | | | | |
| G1/G2 | 53 | 32 (60.4) | 21 (39.6) | 0.19^{a} |
| G3 | 78 | 56 (71.8) | 22 (28.2) | |
| Histotype | | | | |
| Serous | 93 | 66 (71.0) | 27 (29.0) | |
| Mucinous | 12 | 5 (41.7) | 7 (58.3) | |
| Endometrioid | 6 | 5 (83.3) | 1 (16.7) | 0.089^{b} |
| Clear Cell | 4 | 1 (25.0) | 3 (75.0) | |
| Undifferentiated | 11 | 7 (63.6) | 4 (36.4) | |
| x | 5 | | | |
| Residual tumor (cm) | | | | |
| 0 | 68 | 39 (57.4) | 29 (42.6) | |
| ≦2 | 37 | 30 (81.1) | 7 (18.9) | 0.026^{b} |
| >2 | 22 | 17 (77.3) | 5 (22.7) | |
| x | 4 | | | |
| Debulking success^{c} | | | | |
| SD | 59 | 47 (79.7) | 12 (20.3) | 0.008^{a} |
| OD | 68 | 39 (57.4) | 29 (42.6) | |
| x | 4 | | | |
| Acites fluid (ml) | | | | |
| 0 | 41 | 25 (61.0) | 16 (39.0) | |
| ≦500 | 43 | 28 (65.1) | 15 (34.9) | 0.27^{b} |
| >500 | 43 | 33 (76.7) | 10 (23.3) | |
| x | 4 | | | |

¹cutoff used was equal to the 67^{th} percentile (0.13 ng/mg protein)
^{a}Fisher's Exact Test
^{b}χ² test.
^{c}OD; Optimal debulking (0-1 cm), SO; Suboptimal debulking (>1 cm)
x. Status unknown.

TABLE 4
Univariate and multivariate analysis of hK13 status with regard to
progression-free and overall survival

| | Progression-free | | | | | |
|---|---|---|---|---|---|---|
| | survival | | | Overall survival | | |
| Variable | HR^{a} | 95% CI^{b} | p value | HR^{a} | 95% CI^{b} | p value |

Univariate analysis

| | | | | | | |
|---|---|---|---|---|---|---|
| hK13 | | | | | | |
| (N = 131) | | | | | | |
| negative | 1.00 | | | 1.00 | | |
| positive | 0.46 | 0.25-0.82 | 0.009 | 0.33 | 0.15-0.69 | 0.004 |
| as a continuous | 0.99 | 0.89-1.12 | 0.99 | 0.83 | 0.61-1.14 | 0.26 |
| variable | | | | | | |
| Stage of | 2.14 | 1.50-3.04 | <0.001 | 3.03 | 1.95-4.67 | <0.001 |
| disease | | | | | | |
| (ordinal) | | | | | | |
| Grading | 1.42 | 1.04-1.94 | 0.027 | 1.55 | 1.08-2.18 | 0.017 |
| (ordinal) | | | | | | |
| CA125^{c} | 0.97 | 0.91-1.04 | 0.42 | 0.98 | 0.91-1.05 | 0.59 |
| Age | 1.02 | 0.99-1.03 | 0.084 | 1.025 | 1.00-1.05 | 0.029 |

Multivariate analysis^{d}

| | | | | | | |
|---|---|---|---|---|---|---|
| hK13 | | | | | | |
| (N = 120) | | | | | | |
| negative | 1.00 | | | 1.00 | | |
| positive | 0.53 | 0.29-0.97 | 0.042 | 0.34 | 0.15-0.76 | 0.010 |
| as a continuous | 1.00 | 0.91-1.12 | 0.090 | 0.89 | 0.67-1.16 | 0.22 |
| variable | | | | | | |
| Stage of | 2.02 | 1.36-2.98 | <0.001 | 3.37 | 1.99-5.71 | <0.001 |
| disease | | | | | | |
| (ordinal) | | | | | | |
| Grading | 1.13 | 0.81-1.57 | 0.48 | 1.21 | 0.81-1.80 | 0.36 |
| (ordinal) | | | | | | |
| CA125 | 1.00 | 0.93-1.08 | 0.71 | 1.03 | 0.93-1.13 | 0.56 |
| Age | 1.00 | 0.98-1.02 | 0.45 | 1.02 | 0.99-1.04 | 0.19 |

^{a}Hazard ratio (HR) estimated from Cox proportional hazard regression model
^{b}Confidence interval of the estimated HR
^{c}tissue CA125 in KU/mg total protein
^{d}Multivariate models were adjusted for stage of disease, tumor grade, CA125 and age.

TABLE 5
Kallikrein expression (mRNA and protein) in ovarian cancer tissues

| | | |
|---|---|---|
| Kallikrein | Prognosis | Reference |

mRNA¹

| | | |
|---|---|---|
| KLK4 | unfavorable | 96, 97 |
| KLK5 | unfavorable | 98 |
| KLK7 | unfavourable | 100 |
| KLK8 | favorable | 92 |
| KLK9 | favorable | 93 |
| KLK14 | favorable | 95 |
| KLK15 | unfavorable | 102 |

Protein²

| | | |
|---|---|---|
| hK5 | unfavorable | our unpublished data |
| hK6 | unfavorable | 99 |
| hK10 | unfavorable | 101 |
| hK11 | favorable | 94 |
| hK13 | favorable | present study |

¹RT-PCR methodology
²ELISA methodology

### FULL CITATIONS FOR REFERENCES REFERRED TO IN THE SPECIFICATION

### References

1. Bhoola, K. D., Figueroa, C. D., and Worthy, K. Bioregulation of kinins: kallikreins, kininogens, and kininases. Pharmacol Rev 1992;44: 1-80.
2. Clements, J. The molecular biology of the kallikreins and their roles in inflammation. In: S. Farmer (ed.) The Kinin System, pp. 71-97. New York: Academic Press, 1997.
3. Riegman, P. H., Vlietstra, R. J., Suurmeijer, L., Cleutjens, C. B., and Trapman, J. Characterization of the human kallikrein locus. Genomics 1992;14: 6-11.
4. McCormack, R. T., Rittenhouse, H. G., Finlay, J. A., Sokoloff, R. L., Wang, T. J., Wolfert, R. L., Lilja, H., and Oesterling, J. E. Molecular forms of prostate-specific antigen and the human kallikrein gene family: a new era. Urology 1995;45: 729-44.
5. Diamandis, E. P., Yousef, G. M., Clements, J., Ashworth, L. K, Yoshida, S., Egelrud, T., Nelson, P. S., Shiosaka, S., Little, S., Lilja, H., Stenman, U. H., Rittenhouse, H. G., and Wain, H. New nomenclature for the human tissue kallikrein gene family. Clin Chem 2000;46: 1855-8.
6. Yousef, G. M. and Diamandis, E. P. The new human tissue kallikrein gene family: structure, function, and association to disease. Endocr Rev 2001;22: 184-204.
7. Yousef, G. M., Chang, A., and Diamandis, E. P. Identification and characterization of KLK-L4, a new kallikrein-like gene that appears to be down-regulated in breast cancer tissues. J Biol Chem 2000;275: 11891-8.
8. Diamandis E P, Yousef G M, Soosaipillai A R, Grass L, Porter A, Little S, Sotiropoulou G. Immunofluorometric assay of human kallikrein 6 (zyme/protease M/neurosin) and preliminary clinical applications. Clin Biochem 2000;33:369-375.
9. Luo, L Y, Grass L, Howarth D J C, Thibault P, Ong H, Diamandis E P. Immunofluorometric assay of human kallikrein 10 and its identification in biological fluids and tissues. Clin Chem 2001;47:237-246.
10. Diamandis, E. P., Yousef, G. M, Soosaipillai, A. R, Bunting, P. Human kallikrein 6 (zyme/protease M/neurosin): A new serum biomarker of ovarian carcinoma. Clin Biochem 2000;33:579-583.
11. Luo L-Y, Bunting P, Scorilas A, Diamandis E P. Human kallikrein 10: a novel tumor marker for ovarian carcinoma? Clin Chim Acta 2001;7:806-811.
12. Diamandis E P, Okui A, Mitsui S, Luo L Y, Soosaipillai A, Grass L, Nakamura T, Howarth D J, Yamaguchi N. Human kallikrein 11: A new biomarker of prostate and ovarian carcinoma. Cancer Res 2002;62:293-300.
13. Sambrook, J., Fritsch, E. F., and Maniatis, T. Molecular Cloning: A Laboratory Manual, Second Edition edition. NY: Cold Sping Harbor Laboratory, 1989.
14. Shevchenko, A., Wilm, M., and Mann, M. Peptide sequencing by mass spectrometry for homology searches and cloning of genes. J Protein Chem 1997;16: 481-90.
15. Christopoulos, T. K. and Diamandis, E. P. Enzymatically amplified time-resolved fluorescence immunoassay with terbium chelates. Anal Chem 1992;64: 342-6.
16. Stenman, U. H., Leinonen, J., Alfthan, H., Rannikko, S., Tuhkanen, K., and Alfthan, O. A complex between prostate-specific antigen and alpha 1-antichymotrypsin is the major form of prostate-specific antigen in serum of patients with prostatic cancer: assay of the complex improves clinical sensitivity for cancer. Cancer Res 1991;51: 222-6.
17. Lilja, H., Christensson, A., Dahlen, U., Matikainen, M. T., Nilsson, O., Pettersson, K., and Lovgren, T. Prostate-specific antigen in serum occurs predominantly in complex with alpha 1-antichymotrypsin. Clin Chem 1991;37: 1618-25.
18. Stephan C, Jung K, Lein M, Sinha P, Schnorr D, Loening S A. Molecular forms of prostate-specific antigen and human kallikrein 2 as promising tools for early diagnosis of prostate cancer. Cancer Epidemiol Biomark Prev 2000;9:1133-1147.
19. Saedi M S, Zhu Z, Marker K, Liu R S, Carpenter P M, Rittenhouse H. Mikolajczyk S D. Human kallikrein 2 (hK2), but not prostate-specific antigen (PSA), rapidly complexes with protease inhibitor 6 (PI-6) released from prostate carcinoma cells. Int J Cancer 2001;94:558-563.
20. Zhang, W. M., Finne, P., Leinonen, J., Vesalainen, S., Nordling, S., Rannikko, S., and Stenman, U. H. Characterization and immunological determination of the complex between prostate-specific antigen and alpha2-macroglobulin. Clin Chem 1998;44: 2471-9.
21. Zhou, A. M., Tewari, P. C., Bluestein, B. I., Caldwell, G. W., and Larsen, F. L. Multiple forms of prostate-specific antigen in serum: differences in immunorecognition by monoclonal and polyclonal assays. Clin Chem 1993;39: 2483-91.
22. Grauer, L. S., Finlay, J. A., Mikolajczyk, S. D., Pusateri, K. D., and Wolfert, R. L. Detection of human glandular kallikrein, hK2, as its precursor form and in complex with protease inhibitors in prostate carcinoma serum. J Androl 1998;19: 407-11.
23. Grauer, L. S., Charlesworth, M. C., Saedi, M. S., Finlay, J. A., Liu, P. S., Kuus-Reichel, K., Young, C. Y., and Tindall, D. J. Identification of human glandular kallikrein hK2 from LNCaP cells. J Androl 1996;17: 353-9.
24. Deperthes, D., Chapdelaine, P., Tremblay, R. R., Brunet, C., Berton, J., Hebert, J., Lazure, C., and Dube, J. Y. Isolation of prostatic kallikrein hK2, also known as hGK-1, in human seminal plasma. Biochim Biophys Acta 1995;1245: 311-6.
25. Mikolajczyk, S. D., Millar, L. S., Kumar, A., and Saedi, M. S. Prostatic human kallikrein 2 inactivates and complexes with plasminogen activator inhibitor-1. Int J Cancer 1999;81: 438-42.
26. Yousef G M, Diamandis E P. Kallikreins, steroid hormones and ovarian cancer: is there a link? Minerva Endocrinol 2002;27:157-166.
27. Jemal A, Murray T, Samuels A, et al: Cancer statistics, 2003. CA Cancer J Clin 53:5-26, 2003.
28. Auersperg N, Edelson M I, Mok S C, et al: The biology of ovarian cancer. Semin Oncol 25:281-304, 1998
29. Schink J C: Current initial therapy of stage III and IV ovarian cancer: challenges for managed care. Semin Oncol 26:2-7, 1999
30. Trope C: Prognostic factors in ovarian cancer. Cancer Treat Res 95:287-352, 1998
31. Eisenhauer E A, Gore M, Neijt J P: Ovarian cancer: should we be managing patients with good and bad prognostic factors in the same manner? Ann Oncol 10 Suppl 1:9-15, 1999
32. Ozalp S, Yalcin O T, Gulbas Z, et al: Effect of cellular DNA content on the prognosis of epithelial ovarian cancers. Gynecol Obstet Invest 52:93-7, 2001
33. Milde-Langosch K, Hagen M, Bamberger A M, et al: Expression and Prognostic Value of the Cell-cycle Regulatory Proteins, Rb, p16MTS 1, p21WAF1, p27KIP1, Cyclin E, and Cyclin D2, in Ovarian Cancer. Int J Gynecol Pathol 22:168-74, 2003
34. Masciullo V, Ferrandina G, Pucci B, et al: p27Kip1 expression is associated with clinical outcome in advanced epithelial ovarian cancer: multivariate analysis. Clin Cancer Res 6:4816-22, 2000
35. Gotlieb W H, Goldberg I, Weisz B, et al: Topoisomerase II immunostaining as a prognostic marker for survival in ovarian cancer. Gynecol Oncol 82:99-104, 2001
36. Shen G H, Ghazizadeh M, Kawanami 0, et al: Prognostic significance of vascular endothelial growth factor expression in human ovarian carcinoma. Br J Cancer 83:196-203, 2000
37. Hata K, Fujiwaki R, Nakayama K, et al: Expression of the Endostatin gene in epithelial ovarian cancer. Clin Cancer Res 7:2405-9, 2001
38. Schmalfeldt B, Prechtel D, Harting K, et al: Increased expression of matrix metalloproteinases (MP)-2, MMP-9, and the urolinase-type plasminogen activator is associated with progression from benign to advanced ovarian cancer. Clin Cancer Res 7:2396-404, 2001
39. Schummer M, Ng W V, Bumgarner R E, et al: Comparative hybridization of an array of 21,500 ovarian cDNAs for the discovery of genes overexpressed in ovarian carcinomas. Gene 238:375-85, 1999
40. Welsh J B, Zarrinkar P P, Sapinoso L M, et al: Analysis of gene expression profiles in normal and neoplastic ovarian tissue samples identifies candidate molecular markers of epithelial ovarian cancer. Proc Natl Acad Sci USA 98:1176-81, 2001
41. Haviv I, Campbell I G: DNA microarrays for assessing ovarian cancer gene expression. Mol Cell Endocrinol 191:121-6, 2002
42. Sawiris G P, Sherman-Baust C A, Becker K G, et al: Development of a highly specialized cDNA array for the study and diagnosis of epithelial ovarian cancer. Cancer Res 62:2923-8, 2002
43. Hough C D, Sherman-Baust C A, Pizer E S, et al: Large-scale serial analysis of gene expression reveals genes differentially expressed in ovarian cancer. Cancer Res 60:6281-7, 2000
44. Yousef G M, Diamandis E P: The new human tissue kallikrein gene family: structure, function, and association to disease. Endocr Rev 22:184-204, 2001
45. Yousef G M, Diamandis E P: Kallikreins, steroid hormones and ovarian cancer: is there a link? Minerva Endocrinol 27:157-66, 2002
46. Diamandis E P, Yousef G M: Human tissue kallikreins: a family of new cancer biomarkers. Clin Chem 48:1198-205, 2002
47. Diamandis E P, Yousef G M, Soosaipillai A R, et al: Human kallikrein 6 (zyme/protease M/neurosin): a new serum biomarker of ovarian carcinoma. Clin Biochem 33:579-83, 2000
48. Diamandis E P, Scorilas A, Fracchioli S, et al: Human Kallikrein 6 (hK6): A new potential serum biomarker for diagnosis and prognosis of ovarian carcinoma. J Clin Oncol 21:1035-1043, 2003
49. Luo L Y, Bunting P, Scorilas A, et al: Human kallikrein 10: a novel tumor marker for ovarian carcinoma? Clin Chim Acta 306:111-8, 2001
50. Luo L Y, Katsaros D, Scorilas A, et al: The serum concentration of human kallikrein 10 represents a novel biomarker for ovarian cancer diagnosis and prognosis. Cancer Res 63:807-11, 2003
51. Diamandis E P, Okui A, Mitsui S, et al: Human kallikrein 11: a new biomarker of prostate and ovarian carcinoma. Cancer Res 62:295-300, 2002
52. Yousef G M, Chang A, Diamandis E P: Identification and characterization of KLK-L4, a new kallikrein-like gene that appears to be down-regulated in breast cancer tissues. J Biol Chem 275:11891-8, 2000
53. Chang A, Yousef G M, Scorilas A, et al: Human kallikrein gene 13 (KLK13) expression by quantitative RT-PCR: an independent indicator of favourable prognosis in breast cancer. Br J Cancer 86:1457-64, 2002
54. Chang A, Yousef G M, Jung K, et al: Identification and molecular characterization of five novel kallikrein gene 13 (KLK13; KLK-L4) splice variants: differential expression in the human testis and testicular cancer. Anticancer Res 21:3147-52, 2001
55. Kapadia C, Chang A, Sotiropoulou G, et al: Human kallikrein 13: production and purification of recombinant protein and monoclonal and polyclonal antibodies, and development of a sensitive and specific immunofluorometric assay. Clin Chem 49:77-86, 2003
56. Petraki C D, Karavana V N, Diamandis E P: Human Kallikrein 13 Expression in Normal Tissues. An immunohistochemical study. J Histochem Cytochem 51:493-501, 2003
57. Pettersson F: Annual report on the treatment in gynecological cancer. Stockholm, International Federation of Gynecology and Obstetrics, 1994
58. Day T G, Jr., Gallager H S, Rutledge F N: Epithelial carcinoma of the ovary prognostic importance of histologic grade. Natl Cancer Inst Monogr 42:15-21, 1975
59. Serov S F, Sorbin L H: Histological typing of ovarian tumors., World Health Organization., 1973
60. Christopoulos T K, Diamandis E P: Enzymatically amplified time-resolved fluorescence immunoassay with terbium chelates. Anal Chem 64:342-6, 1992
61. Cox D R: Regression models and life tables. R—Stat. Soc. B. 34:187-202, 1972
62. Kaplan E L, Meier P: Nonparametric estimation from incomplete observations. J. Arm. Stat. Assoc. 53:457-481, 1958
63. Mantel N: Evaluation of survival data and two new rank order statistics arising in its consideration. Cancer Chemother Rep 50:163-70, 1966
64. Duffy M J: Proteases as prognostic markers in cancer. Clin Cancer Res 2:613-8, 1996
65. Duffy M J, Maguire T M, McDermott E W, et al: Urokinase plasminogen activator: a prognostic marker in multiple types of cancer. J Surg Oncol 71:130-5, 1999
66. Vihinen P, Kahari V M: Matrix metalloproteinases in cancer: prognostic markers and therapeutic targets. Int J Cancer 99:157-66, 2002
67. Kos J, Lah T T: Cysteine proteinases and their endogenous inhibitors: target proteins for prognosis, diagnosis and therapy in cancer (review). Oncol Rep 5:1349-61, 1998
68. Harbeck N, Alt U, Berger U, et al: Prognostic impact of proteolytic factors (urokinase-type plasminogen activator, plasminogen activator inhibitor 1, and cathepsins B, D, and L) in primary breast cancer reflects effects of adjuvant systemic therapy. Clin Cancer Res 7:2757-64, 2001
69. Seetoo D Q, Crowe P J, Russell P J, et al: Quantitative expression of protein markers of plasminogen activation system in prognosis of colorectal cancer. J Surg Oncol 82:184-93, 2003
70. Foekens J A, Ries C, Look M P, et al: The prognostic value of polymorphonuclear leukocyte elastase in patients with primary breast cancer. Cancer Res 63:337-41, 2003
71. Ghosh S, Wu Y, Stack M S: Ovarian cancer-associated proteinases. Cancer Treat Res 107:331-51, 2002
72. Konecny G, Untch M, Pihan A, et al: Association of urokinase-type plasminogen activator and its inhibitor with disease progression and prognosis in ovarian cancer. Clin Cancer Res 7:1743-9, 2001
73. Lengyel E, Schmalfeldt B, Konik E, et al: Expression of latent matrix metalloproteinase 9 (MMP-9) predicts survival in advanced ovarian cancer. Gynecol Oncol 82:291-8, 2001
74. Wu X, Li H, Kang L, et al: Activated matrix metalloproteinase-2—a potential marker of prognosis for epithelial ovarian cancer. Gynecol Oncol 84:12&34, 2002
75. Sui L, Dong Y, Ohno M, et al: Survivin expression and its correlation with cell proliferation and prognosis in epithelial ovarian tumors. Int J Oncol 21:315-20, 2002
76. Duffy M J: The role of proteolytic enzymes in cancer invasion and metastasis. Clin. Exp. Metastasis 10:145-155, 1991
77. Del Rosso M, Fibbi G, Pucci M, et al: Multiple pathways of cell invasion are regulated by multiple families of serine proteases. Clin Exp Metastasis 19:193-207, 2002
78. Noel A, Gilles C, Bajou K, et al: Emerging roles for proteinases in cancer. Invasion Metastasis 17:221-39, 1997
79. DeClerck Y A, Imren S, Montgomery A M, et al: Proteases nd protease inhibitors in tumor progression. Adv Exp Med Biol 425:89-97, 1997
80. Aimes R T, Zijlstra A, Hooper J D, et al: Endothelial cell serine proteases expressed during vascular morphogenesis and angiogenesis. Thromb Haemost 89:561-72, 2003
81. Impola U, Toriseva M, Suomela S, et al: Matrix metalloproteinase-19 is expressed by proliferating epithelium but disappears with neoplastic dedifferentiation. Int J Cancer 103:709-16, 2003
82. Hooper J D, Nicol D L, Dickinson J L, et al: Testisin, a new human serine proteinase expressed by premeiotic testicular germ cells and lost in testicular germ cell tumors. Cancer Res 59:3199-205, 1999
83. Chen L M, Chai K X: Prostasin serine protease inhibits breast cancer invasiveness and is transcriptionally regulated by promoter DNA methylation. Int J Cancer 97:323-9, 2002
84. Takahashi S, Suzuki S, Inaguma S, et al: Down-regulated expression of prostasin in high-grade or hormone-refractory human prostate cancers. Prostate 54:187-93, 2003
85. Chen L M, Hodge G B, Guarda L A, et al: Down-regulation of prostasin serine protease: a potential invasion suppressor in prostate cancer. Prostate 48:93-103, 2001
86. Balbay M D, Pettaway C A, Kuniyasu H, et al: Highly metastatic human prostate cancer growing within the prostate of athymic mice overexpresses vascular endothelial growth factor. Clin Cancer Res 5:783-9, 1999
87. Goyal J, Smith K M, Cowan J M, et al: The role for NES1 serine protease as a novel tumor suppressor. Cancer Res 58:4782-6, 1998
88. Scorilas A, Diamandis E P, Levesque M A, et al: Immunoenzymatically determined pepsinogen C concentration in breast tumor cytosols: an independent favorable prognostic factor in node-positive patients. Clin Cancer Res 5:1778-85, 1999
89. Scorilas A, Karameris A, Amnogiannaki N, et al: Overexpression of matrix-metalloproteinase-9 in human breast cancer: a potential favourable indicator in node-negative patients. Br J Cancer 84:1488-96, 2001
90. Rojo J V, Merino A M, Gonzalez L O, et al: Expression and clinical significance of pepsinogen C in epithelial ovarian carcinomas. Eur J Obstet Gynecol Reprod Biol 104:58-63, 2002
91. Hsia J Y, Chen C Y, Chen J T, et al: Prognostic significance of caspase-3 expression in primary resected esophageal squamous cell carcinoma. Eur J Surg Oncol 29:44-8, 2003
92. Magklara A, Scorilas A, Katsaros D, et al: The human KLK8 (neuropsin/ovasin) gene: identification of two novel splice variants and its prognostic value in ovarian cancer. Clin Cancer Res 7:806-11, 2001
93. Yousef G M, Kyriakopoulou L G, Scorilas A, et al: Quantitative expression of the human kallikrein gene 9 (KLK9) in ovarian cancer: a new independent and favorable prognostic marker. Cancer Res 61:7811-8, 2001
94. Borgono C A, Fracchioli S, Yousef G M, et al: Favourable Prognostic Value of Tissue Human Kallikrein 11 (hK11) in Patients with Ovarian Carcinoma. International Journal of Cancer in press, 2003
95. Yousef G M, Fracchioli S, Scorilas A, et al: Steroid hormone regulation and prognostic value of the human kallikrein gene 14 in ovarian cancer. Am J Clin Pathol 119:346-55, 2003
96. Obiezu C V, Scorilas A, Katsaros D, et al: Higher human kallikrein gene 4 (KLK4) expression indicates poor prognosis of ovarian cancer patients. Clin Cancer Res 7:2380-6, 2001
97. Dong Y, Kaushal A, Bui L, et al: Human kallikrein 4 (KLK4) is highly expressed in serous ovarian carcinomas. Clin Cancer Res 7:2363-71, 2001
98. Kim H, Scorilas A, Katsaros D, et al: Human kallikrein gene 5 (KLK5) expression is an indicator of poor prognosis in ovarian cancer. Br J Cancer 84:643-50, 2001
99. Hoffman B R, Katsaros D, Scorilas A, et al: Immunofluorometric quantitation and histochemical localisation of kallikrein 6 protein in ovarian cancer tissue: a new independent unfavourable prognostic biomarker. Br J Cancer 87:763-71, 2002
100. Kyriakopoulou L G, Yousef G M, Scorilas A, et al: Prognostic value of quantitatively assessed KLK7 expression in ovarian cancer. Clin Biochem 36:135-43, 2003
101. Luo L Y, Katsaros D, Scorilas A, et al: Prognostic value of human kallikrein 10 expression in epithelial ovarian carcinoma. Clin Cancer Res 7:2372-9, 2001
102. Yousef G M, Scorilas A, Katsaros D, et al: Prognostic value of the human kallikrein gene 15 (KLK15) expression in ovarian cancer. J Clin Oncol (in press, 2003)
103. Henderson B E, Feigelson H S: Hormonal carcinogenesis. Carcinogenesis 21:427-33, 200Liao D J, Dickson R B: Roles of androgens in the development, growth, and carcinogenesis of the mammary gland. J Steroid Biochem Mol Biol 80:175-89, 2002
104. Risch H A: Hormonal etiology of epithelial ovarian cancer, with a hypothesis concerning the role of androgens and progesterone. J Natl Cancer Inst 90:1774-86, 1998
105. Isola J J: Immunohistochemical demonstration of androgen receptor in breast cancer and its relationship to other prognostic factors. J Pathol 170:31-5, 1993
106. Cardillo M R, Petrangeli E, Aliotta N, et al: Androgen receptors in ovarian tumors: correlation with oestrogen and progesterone receptors in an immunohistochemical and semiquantitative image analysis study. J Exp Clin Cancer Res 17:231-7, 1998

1-2. (canceled)

3. A method as claimed in claim 5 wherein the kallikrein 13 is detected using antibodies specifically reactive with kallikrein 13 or a part thereof.

4. (canceled)

5. A method for detecting or screening a subject for endocrine cancer comprising:
(a) obtaining a biological sample from a subject;
(b) detecting the amount of kallikrein 13 in said sample; and
(c) comparing said amount of kallikrein 13 detected to a predetermined standard, where detection of a level of kallikrein 13 that is significantly different than that of a standard is indicative of endocrine cancer.

6. (canceled)

7. A method for monitoring the progression of endocrine cancer in a subject, the method comprising:
(a) detecting in a sample from the subject at a first time point, kallikrein 13;
(b) repeating step (a) at a subsequent point in time; and
(c) comparing levels detected in steps (a) and (b), and thereby monitoring the progression of endocrine cancer.

8. A method of claim 5 for screening a subject for endocrine cancer comprising:
(a) incubating a biological sample from the subject with a first antibody specific for kallikrein 13 which is directly or indirectly labeled with a detectable substance, and a second antibody specific for kallikrein 13 which is immobilized;
(b) detecting the detectable substance thereby quantitating kallikrein 13 in the biological sample; and
(c) comparing the quantitated kallikrein 13 with levels for a predetermined standard.

9. A method of claim 5 for determining the prognosis of a subject with ovarian cancer comprising:
(a) contacting an amount of an antibody which binds to kallikrein 13, with a sample from the subject so as to form a complex comprising the antibody and kallikrein 13 in the sample;
(b) determining or detecting the presence or amount of complex formation in the sample;
(c) comparing the amount of kallikrein 13 present in the sample with the amount of kallikrein 13 in a control, wherein a higher amount of kallikrein 13 in the sample compared with the amount in the control is indicative of a favourable prognosis, in particular, early stage disease, no residual tumor, optimal debulking success, longer PFS and/or OS.

10. A method of claim 5 which further comprises detecting one or more of human stratum corneum chymotryptic enzyme (HSCCE), haptoglobin alpha, osteopontin, kallikrein 2, kallikrein 3, kallikrein 4, kallikrein 5, kallikrein 6, kallikrein 8, kallikrein 10, kallikrein 11, kallikrein 14, kallikrein 15, CA125, CA15-3, CA19-9, OVX1, lysophosphatidic acid (LPA) or carcinoembryonic antigen (CEA).

11. A method for assessing the potential efficacy of a test agent for inhibiting endocrine cancer in a subject, the method comprising comparing: (a) levels of kallikrein 13 in a first sample obtained from a subject and exposed to the test agent; and (b) levels of kallikrein 13 in a second sample obtained from the subject, wherein the sample is not exposed to the test agent, wherein a significant difference in the levels of expression of kallikrein 13 in the first sample, relative to the second sample, is an indication that the test agent is potentially efficacious for inhibiting endocrine cancer in the subject.

12-20. (canceled)

21. A kit for assessing whether a patient subject is afflicted with endocrine cancer, the kit comprising reagents that specifically bind with kallikrein 13.

22. (canceled)

23. A kit as claimed in claim 21 wherein the reagents are antibodies that specifically bind with protein or protein fragments corresponding to kallikrein 13.

24. A method of claim 5 wherein the endocrine cancer is ovarian cancer and the standard comprises normal levels of kallikrein 13 in samples of the same type obtained from control subjects not afflicted with ovarian cancer or who have kallikrein negative tumors.

25. A method of claim 24 wherein the levels of kallikrein 13 in the sample from the subject are increased compared to the standard and are indicative of early stage ovarian cancer, no residual tumors, optimal debulking success, tumors characterized by clear cell and mucinous histotypes, and/or longer survival.

26. A method of claim 5 further comprising detecting kallikrein 5 and/or kallikrein 11.

27. A method of claim 25 wherein the ovarian cancer is non-serous eptithelial tumors.

28. A method of claim 9 wherein the control comprises normal levels of kallikrein 13 in samples of the same type obtained from control subjects not afflicted with ovarian cancer or who have kallikrein negative tumors.

29. A method of claim 28 wherein the subject has a non-serous epithelial tumor.


SEQUENCE LISTING (6 sequences)

SEQ ID NO: 1 - protein, 277 aa, homo sapiens
  ref <308>: DATABASE ACCESSION NUMBER: AF135024
  ref <309>: DATABASE ENTRY DATE: 2000-06-26
  ref <313>: RELEVANT RESIDUES: (1)..(277)
MWPLALVIASLTLALSGGVSQESSKVLNTNGTSGFLPGGYTCFPHSQPWQAALLVQGRLLCGGVLVHPKWVLTAAHCLKEGLKVYLGKHALGRVEAGEQVREVVHSIPHPEYRRSPTHLNHDHDIMLLELQSPVQLTGYIQTLPLSHNNRLTPGTTCRVSGWGTTTSPQVNYPKTLQCANIQLRSDEECRQVYPGKITDNMLCAGTKEGGKDSCEGDSGGPLVCNRTLYGIVSWGDFPCGQPDRPGVYTRVSRYVLWIRETIRKYETQQQKWLKGPQ

SEQ ID NO: 2 - DNA, 10080 bp, homo sapiens
  ref <308>: DATABASE ACCESSION NUMBER: AF135024
  ref <309>: DATABASE ENTRY DATE: 2000-06-26
  ref <313>: RELEVANT RESIDUES: (1)..(10080)
caggaggttgcacactgttcctcccacctcgccactgcacccccaccaaggatggaattggaggcggggggcagattccagggtcagggctgtcaagagtgaatgaggcgaggagacattcaggagcagagaggtttcagacgcggaggttccgggcacgccctcaacacccccttcaccttctcctcaggccccgcccgccctgccctcccctcccgatcccggagccatgtggcccctggccctagtgatcgcctccctgaccttggccttgtcaggaggtaagaatgcgcgggggtggaggcgcggcggccattcgggacaatggtaggaggggtcaggccggagggggagggggcgtgggagccgcgagctccgccccccgcccactcggggccgggtccagtggggacagctcagagctcttcctgcttgtccctgggtgacctggtttcccggctgaggttggccctccgaccccagacccttcacctcccaaaataccctcgcagcagcccctcccgcgttcaaggcttcctgtcctctctggaaagctgaaagacatgggttcgcgtcctgacgctgccgctttgagccagtagcctagcagctgctttgtgcctaaattgttttcatctggaaaatgggcttaatctataagtgcttaccagagaaggtcactgtgaatattgaaacgaggtaatgcgtcgagccttcagtatgtcgcaggtagaagggacttgaaagttagccacttagccgttattactttattagtagtattcctttttttttttttttttttttttgagatggaaccttgctctgtctcccaggctggaaggcagtggcacgatcttggcttactgaaacctccgcctcccgggttgaagcgattctcctgcctcagcctcccgagtagctgggattacaggcgcccgccaccacgcccaactaatttttgtattttcagcagagacggggtttcgccatgttggttaggctggtctcgaacttctaacttcaagtagcccgcgtcagcctcccaaagtgccaggattacaggcatgagccaccgagcccggcctctagtattctgtcttcatactcagccccttccagaaccttctagattgttattttaatccttgggttgaccccaaacctatgtgacctcaccccaaattggtagtccttaagatccttatggatctttcccatctttccctgccgttgtaggcaggttctctggaaaccccgttcatgaatcatttattcattcaacaaacagctattaaacaccggccactgtgctgggtgctgtacaagcagagacacagtccctgctctcagcacctggagtctagcggggacagacgcagatgttattcaaacaattatccaaataattagttaataattatcttgacatgaggtgaagacttcaaggagccaagccaggggcctagagatgtaatggcggcttcccgaccagaggccttcccaaagggcttgacccttgagccaagacctgaaaaaggagggatctgtgggtgcctggcacctggcaccatccttggcctgaaggtggggtggcttttctcctctggcgacactccctggattcatgcccgtgccactcctgagtgccacaccctaggctaggagacccacacgctacgccttgtggagtcctcaacaacctggcgaggtaggtgcattgtaattactccaatttcatggcagagaaacctaggactcaaagacagaaggctcctgctccaatgacaccggcgatgcctgagtcagaatcctaatcaaggttgttttccctgtccatatcctggacttgaggctctgaaaaccatttttataacttttgacctaatcatttgcttaaagttagctttttttcttcttttttcactcaaacaaaagcatgttcaactttatattactgtcctgaatagagaatagaattctttgtcataaatagaaggtaaggaaggaaataaatcctgcacaatgaaaagaaaataatatgtttattgggttggaccacctgaaattgctgatacttgaccctttttgaccttcctaaaacaacttttgcagatggttcagtgtaataaatgttaggtggcctgatgaggcttctgtgtcctcctggctttgaaaagtgagctcagtgaggattagggaggtgttaaaaccatattagcaccatcctgagactttatccttgacaaaatcaggtttaaaagagaactggatgctggttcagcgtctgagtgtgcgatttaacgttacttaaatctcatctctctaccatctaaaatgatcctgtgctcaccgacaacttctgtccctaactgcaaaccactgagctaatccaactgcttgccctgtagttggggaaactagctagggaggcagagggacctcctgttgtagctaataattaataataacatttcccactgactgagtgctctccatgccacctgctgtgctgcacggtttgaaatgcaggatcatcttgaattcttcaactgcgcaatgagagatgaactattactttttctacttgacagctggggaaactgaggctggtgatttgcataaggtcacacagtcacaaaatggcatgcatgttcaggattggattctccctgtcccacggacccctgctgtgctttcaatgccagacacagtgcctggcacacacagcatttatttattgagcccccattgtgtgccaggcgctgtgttaggtcctgggaatatggtactgaataaagcagttaaggtgcctgttgtcaatggagcttacagtcaaagtggagagatttttaaaaacgaatacatacaaatgtgaagagaaatgaatagcaatcattgttctgatgaagaccaactggaagaatgtaatgggggaggagtcgggaccaggagagtcaacattagaccaggtggtcagggaaggcctttctgaagaggagacatttgagctgacctctcagaattaagaaggacccagacatacaacctctaaattctgagggtcatccagtagaatattccatatatgtatatatgaaatatcctatatctgtgctgtccaattatccactagccccttcaggctattgaacatttgaaatatggctggtgtgacttaagaactgaatttttaatttagttttacttcattttaattagtttaaatttaaatagccacatgtagctagtggctaccatattaaacaacataggtctggagaaaggactgtgcagagagaggaaatagcaagtataaaatgtctagtatgggggcatccaagatgatttaaattcttcttttctttaaatgcctggtgtgtttgaagaacaggcccatgaggctggactagaggaagtcagaagaaagaggttggagatggggtcaaagaggctggcaagggccagacagcacagagtcctgcacaccttgggaaggctttttggattttattttaaagaaagttgagcctgggaacaacatctgactttctttgtttgaagagtcctcagcctactttgagaagactggatcggagggatgtaaaagtggaaggatttaggttaatgttgtagtcatttgggctacagaagatggggcatggaccaagatggtggcagaagtgtggagataactggatatttgggagataaaaccaataggaactggttgtgagtgatgaaggaaagaagagaagcaaagatgactcccaggtttggggctgagcactgaggtgggaaatactggagcgaacagttttgattgagaagaatcaagttgggaatacaaagcttaagatgcctgtaaggcatccaaatcaacagtgtttgagttttgagcttaaagaagagttcagggctggagatgattagcctatagctggtatttaaagccatggaggcaaccagtatatatgcagtgaaaggatagagagatgggtggaaagatgattggatggatgcatggatggatatatggatagatggatggatggatggttggattggatggatggatggatggatggatggatggatggatggatggatggatgaataaatggaccagtggatggagggacagatgagtggatggatggttggatggatggatggatggatggatggatagatggttagatgactacctaaatggatgaatggatagatggatgagtagacggatggacaaatcaataggatgaatgggggatggatgattggatagattgatggatagatattgcctaggtggatgtgtaggtcagtctcacttctacctcctgaaatccatcttctggtagaatgatataaaaaatgcatgtggagagaaagtcaggctcctgcttacctatcagcaacatcctcattttgtgaactcttctgttaacccccagtggaggatttggtacttcctgagaaaataatgtcacccctttgccctaattcatctccacttggtcaagaatagcaactgccataggtcggcaaattcatcttcagttcctggtcacccagggcaataatccgacccttaccccaaacccagaaaccacaaccccagggctcctctgccccctggatcccagttttctaacaatctctcttctttaccaggtgtctcccaggagtcttccaaggttctcaacaccaatgggaccagtgggtttctcccaggtggctacacctgcttcccccactctcagccctggcaggctgccctactagtgcaagggcggctactctgtgggggagtcctggtccaccccaaatgggtcctcactgccgcacactgtctaaaggagtatgtgggggccgggggagcatggggtagggatgagaatgggactgggattgtggatggggtagagttggatttgaggatggagttggagttagggttggggatggacatgggagtgagaatgaggtttggggttgagatatggggattgggtatgggaatagaatcaaagtaggggatttggatgggattgaagttgaggatgggggagatgtatttggagatgaggaaggtaggatggagaagaagttaggttggggatgggaagaggttggggctgggatggggatggaaatgggctcatcttctttcctaaccaccttctttctgcacccacagggggctcaaagtttacctaggcaagcacgccctagggcgtgtggaagctggtgagcaggtgagggaagttgtccactctatcccccaccctgaataccggagaagccccacccacctgaaccacgaccatgacatcatgcttctggagctgcagtccccggtccagctcacaggctacatccaaaccctgcccctttcccacaacaaccgcctaacccctggcaccacctgtcgggtgtctggctggggcaccaccaccagcccccagggtatgcacccacacaggtggcctgaggccccataggagtggctggggaaacaggggcagagatgggagggaaggtctgaggtaggttcctttatatataaaaatataaataagtaaataaatatatatatttaaagttagctgtatcctttatataaatataaattcatgaatatataaaaatatgagtatataaattcatgaatatatagaaatataaatagatctaatatatgaatatattatatgatgtatattatgtattatatagtaatataattatatattatacaaaaagtatacaaattaaatgtattttataaattataaaatttatcaattatgtattttaaatatgtatttctgcataatgtatatattatatataatctatatttaaattatatattataaatgtattttataaatgtatacatttatatatttatatactgtaaatgaattttatcatttataatatataaatcatacatataaaatgtttatatttctataatttataaaatgtttaatatattaaatatggttattaatgaaatgtctaataattcaatgtaataattaattctatatcattacttagtaagtataatacattatatatgtgaatataaagttgatgtatataccgacaagagccctttgcatctccctagcaatccctgactctctcccagcctcatgtttgtatctttctcctcaacatgccctgtctctcttcctaccattctatccaactctcccgtaactcttcccatccctgttcctgcttttcccatctttaattctctatttctgaccatctccctattccaactccctctctccaactttctctccccaccgctggctccaccactctccttatcaaccttccattctcttgtcccttccctccttgtccttccctccacttttctcctcatctctcccttcgcctctctcccatgtccctccatatttctgtcacttccgttgctttacccagataggtgctcatctcttctcccatctttctcttcccatctcaattttctatctactctttacccattcaactcgcctatttcaccttcatcccatatcctatccaggtcggataccttagaccttctctttcttctccccagtgaattaccccaaaactctacaatgtgccaacatccaacttcgctcagatgaggagtgtcgtcaagtctacccaggaaagatcactgacaacatgttgtgtgccggcacaaaagagggtggcaaagactcctgtgaggtgaggccgggaggctggtgggtgccttggacaggatagaaagccagaatggaagtgacagatgctggggaaaaagctttgtttccagccttaggggaaccaatctttataagatacaatgtcccctcacataggaggtcaagacaaaaaggggtacccagggatggcaggaataattcatcataagccccagctttgactgagtggctgccaagatccctgtgttgagatgcataaaggttggtattctttcacttgtgagtgatagacaaccaactcaaactggcttaaacaaaatgcaggcttttgtaactgaaaatccaggttgtctggctttaggcacagatggatccaggtatgcaaattgtgtgtttggaattctgtctttcttttaactctcagctcttctttattctgttttggcttcattctcggttagattcttcccatgacaagatggccccagcagctttgagcttacatcctaccctctaggcaaccctattagaaagagaacctctcttttccaatagttcacacaaaagtcttaagcatgattctcactaggctgacctaagtcatgtgtcttgagccatcactccaccagagctgtgggattctctgatgggccaagcctgagtcacatagttaactgtgggtgctggagaggggcagggacaaactgcatggattggaagtggagaagggcagttccccaaatgaaaaaatcaggagaggctgttaccaaaataaggggaaatggccaagtacagtagttcatgcctgtaatcccagcactttgggaggctgaggtgagaggattacttgagcccaggagtttgagaccagcctgggcaacatagtgagactctgtctctacaaaaagaaaaaaaagtttttaaattagccaggtgtggtggagtacaactgcagtcctagttactcgggaggctgaggcagaaggactatttgaacccaggagttcaaggctgcagtgaggtatgatcatgccactgcactccagcctgggtgatagagcaaggccctgtctctaaaacaaaaagaaataaatagagcaagacactgtctctaataaataaataaataaaaatttaaaaatgaatgtttaattttttaaaaataagaggaaatggatactacatgagcaaaaaatagccttcatcaataaagaagttgagattggattcagtgagaaagagtatgatactatattaatgatatgtgccttgatcgattagtgatgtctgccttgggcccaggaagagaaatagacttacacgtgtgttgcataccctgcccagatatgaatgggttcactcaatagtgagagacacaaatgagccttaaataggagcagggtcagctggtgtggggcagggggtgatttagtaccagggaaacaaaaatgggtatgaagtaagttgttaccattttaatgaaactgaggaacagagaaaaacacagaaatttctctgtgtctctctttctctgggcctatctctgtctttctgtccctatttctgtctcttgctgtctgtccctctgtgtttgtcttcttgtctgtttctcactgtcttcattgctttctctcacactgtgtgtgtctgactctgcctctctgagtctccttctctgtgtgtgtctctctccatctttcactctctccccacacctccctgtccctgccttgtttagccccagcaaggacccacctctctctctctttctttccccaactcagggtgactctgggggccccctggtctgtaacagaacactgtatggcatcgtctcctggggagacttcccatgtgggcaacctgaccggcctggtgtctacacccgtgtctcaagatacgtcctgtggatccgtgaaacaatccgaaaatatgaaacccagcagcaaaaatggttgaagggcccacaataaaagttgagaaatgtaccggcttccatcctgtcaccatgacttcctcacatggtctgcttagcccttctctgctccttattcccagtgttccatttgaaccagtgatccatgtcctgaaaaatgctcaatctcagctaacattccatgtttcagaagcattcaggcactgccaggcttgcagtctcccagatgttgcatccctgaaacatctcaacaacctgaatgtcccaacccagacaatggcccaggtctctcaacttcatcagtgtggcttctatgagcccagatcaccacctgaacgttctgtctgtggcacattcttaaatatttccatcagcccatctcaacaatatatgtcctataaatggaccatccttgacaacatcctctaactcttcaagtatttattcaatgccagtatcctagaccttctattttttgcactcaagaaggctctagactcccatgatagttcatcctgaaaatattctcttatgcccacaatcttctgccctgacaacattctgtgtacctctgtgactcaccacagctaacattggatcctcagaatatttcattctcacactgttatgggtgtctcagaagtcccaacccaacctacatcccacattcttccaataccccacctctgccaacattccctctctgaatcaatggcaccctagtctctagagttatagggttcagtataccaaagggtcttcttgcctgaactttattgtctaccaaatattccgtcttgtatcccctccatgaacatccttggtcagtgtcccttgctgttacatctttgtgcatgaccctaaaatgtagtgcaaatccttgctttggacaagttataaaactcacagtctctgtgctttctcatctgtaaaatgggttcataattttttttaattgtaacattattacaagaataaatgtcaagcatttatcactattattatttgcatggttcccataaaatattaccttagaatgttaataacagcccttcgaatttgcagagtgtccaaaaaaagtgttgcactgatttattttcctcaggagacatttcttcagtgttgactatgtgcaagcactctcctgggtgttgttaaatatagtttatttactcaacaaatatttgtacctatcaagagccaggcactgttgcagagacaagtgataaccaatgagttaaacagataaaaacttctgcccttgtagaacttacattcttttcaagaagtctccataacaatgaataaagaaataggctgtcaggtggtgctgcaagccatagcaagaaatgaaacaagggccatatgtggtagctcatgcctgtaataccaacactgggaggccaa

SEQ ID NO: 3 - DNA, 27 bp, artificial
  Primer
tccaaggaattcaacaccaatgggacc

SEQ ID NO: 4 - DNA, 27 bp, artificial
  primer
ccattgtctagattgggacattcaggt

SEQ ID NO: 5 - protein, 16 aa, homo sapiens
VSGWGTTTSPQVNYPK

SEQ ID NO: 6 - protein, 6 aa, homo sapiens
VLNTNG